(19) United States
(12) Patent Application Publication
(10) Pub. No.: US 2026/0151363 A1
(43) Pub. Date: Jun. 4, 2026

(54) MELANOGENESIS INHIBITION COMPOSITIONS AND METHODS OF USE THEREOF

(71) Applicant: MDI BIOLOGICAL LABORATORY, Bar Harbor, ME (US)

(72) Inventors: Romain Madelaine, Bar Harbor, ME (US); Aissette Baanannou, Bar Harbor, ME (US); Romain Menard, Bar Harbor, ME (US)

(73) Assignee: MDI BIOLOGICAL LABORATORY, Bar Harbor, ME (US)

(21) Appl. No.: 19/457,069

(22) Filed: Jan. 22, 2026

Related U.S. Application Data
(63) Continuation of application No. PCT/US2024/039268, filed on Jul. 24, 2024.
(60) Provisional application No. 63/528,800, filed on Jul. 25, 2023.

(51) Int. Cl. A61K 31/15; A61K 8/46; A61K 31/145; A61K 45/06; A61P 35/04; A61Q 19/02

(52) U.S. Cl. CPC A61K 31/15; A61K 8/466; A61K 31/145; A61K 45/06; A61P 35/04; A61Q 19/02

(57) ABSTRACT

The disclosure provides methods for inhibiting: tyrosinase function and/or regulation or its activity, melanogenesis, melanin production, and cell proliferation of melanomas using compounds described here, or analog(s) thereof, or compositions comprising such compounds or analog(s) thereof in a therapeutically effective amount to bind or occupy any one or more of the tyrosinase binding sites in an amount sufficient to inhibit tyrosinase (i.e., function or regulation) or its activity, inhibit melanogenesis, inhibit melanin production, even out skin pigmentation or skin tone, brighten skin pigmentation or skin tone, inhibit cell proliferation of melanomas, and ameliorate or treat skin cancer (e.g., melanoma) in a subject in need thereof.

### CROSS-REFERENCE TO RELATED APPLICATIONS

This application is a continuation under 35 U.S.C. § 111(a) of PCT International Application No. PCT/US2024/039268, filed Jul. 24, 2024, designating the United States and published in English, which claims priority to and the benefit of U.S. Provisional Application No. 63/528,800, filed Jul. 25, 2023, the entire contents of each of which are incorporated by reference herein.

### BACKGROUND

Melanoma, one of the most dangerous forms of skin cancer, represents a significant challenge to traditional cancer therapies due to its characteristic drug resistance. There is an urgent need for new melanoma treatments.

Melanocytes, the cells affected by melanoma, are responsible for synthesizing the pigment, melanin. The process of melanin synthesis, termed “melanogenesis” is relevant to the cosmetics industry because of the demand for treatments for hyperpigmentation, vitiligo, and skin tone brightening. While several melanin synthesis inhibitors are known, such as hydroquinone, arbutin, and kojic acid, these agents have a number of negative physiological effects. Therefore, there is a need for identifying new agents with melanogenesis-inhibiting capabilities for cancer therapies and dermatological, including skin care and cosmetic, use.

### SUMMARY OF THE DISCLOSURE

The embodiments of the disclosure provide methods for inhibiting melanogenesis. Compositions and articles defined by the embodiments of the disclosure were isolated or otherwise manufactured in connection with the examples provided below. Other features and advantages of the disclosure will be apparent from the detailed description, and from the claims.

In one aspect, the disclosure provides a method of inhibiting tyrosinase in a cell, the method including contacting the cell with ML233, or analog(s) thereof, thereby inhibiting tyrosinase in the cell. In another aspect, the present disclosure provides a method of inhibiting melanin production in a cell, the method including contacting the cell with ML233, or analog(s) thereof, thereby inhibiting melanin production in the cell. In embodiments of the methods described here, the cell is a cell expressing tyrosinase. In some embodiments of the methods described here, the cell is a cell expressing melanin. In embodiments of the methods described here, the cell is a melanocyte. In embodiments of the methods described here, the cell is a cell in vivo or in vitro. In embodiments of the methods described here, the cell is a neoplastic cell. In some embodiments of the methods described here, the contacting is by topical or parenteral administration.

In another aspect, the disclosure provides a method for treating a disease associated with the production of melanin in a subject, including administering to the subject a composition including ML233, or analog(s) thereof, thereby treating the disease. In embodiments of the methods described here, the disease is associated with an undesirable increase in pigmentation. In embodiments of the methods described here, the disease is selected from the group consisting of Addison's disease, hyperpigmentation, melasma, and solar lentigo. In certain embodiments of the methods described here, the disease is associated with an undesirable decrease in pigmentation. In such embodiments of the methods described here, the disease is vitiligo or hypopigmentation. In various embodiments of the methods described here, the composition is formulated for topical, oral, or intravenous delivery.

In another aspect, the disclosure provides a method for treating melanoma, the method including contacting the cells with ML233, or analog(s) thereof, thereby treating the melanoma. In embodiments of the methods described here, the method further includes administrating a chemotherapy drug or treating the subject in need thereof with radiation treatment.

In a further aspect, the disclosure provides a method for evening (e.g., lightening, brightening, or reducing) skin pigmentation in a subject, the method including administering to the subject a composition including ML233, or analog(s) thereof, thereby evening (e.g., lightening, brightening, or reducing) skin pigmentation in the subject. In embodiments of the methods described here, the composition further includes one or more physiologically acceptable carriers.

Additional aspects of the disclosure provide methods of using the disclosed compounds, or analog(s) thereof, where the analog(s) is selected from the group consisting of:
[(Z)-(5-methyl-4-oxo-2-propan-2-ylcyclohexa-2,5-dien-1-ylidene)amino]benzenesulfonate (Compound CID: 5731374; PubChem SID: 43 7251522; CSSS00159739712);
[(E)-(5-methyl-4-oxo-2-propan-2-ylcyclohexa-2,5-dien-1-ylidene)amino]4-methylbenzenesulfonate (Compound CID: 5731427; PubChem SID: 437714551;CSSS00160289749);
[(E)-(2-methyl-4-oxo-5-propan-2-ylcyclohexa-2,5-dien-1-ylidene)amino]benzenesulfonate (Compound CID: 5 786741; PubChem SID: 43791245 7; CSSS00160623089);
[(Z)-(5-cyclohexyl-2-methyl-4-oxocyclohexa-2,5-dien- 1-ylidene)amino]benzenesulfonate (Compound CID: 46905036);
[(Z)-(2-methyl-4-oxo-5-propan-2-ylcyclohexa-2,5-dien-1-ylidene)amino]benzenesulfonate (Compound CID: 5601088);
[(E)-(5-methyl-4-oxo-2-propan-2-ylcyclohexa-2,5-dien-1-ylidene)amino]benzenesulfonate (Compound CID: 5650290);
[[4-oxo-3,5-di(propan-2-yl)cyclohexa-2,5-dien-1-ylidene]amino]benzenesulfonate (Compound CID: 5258778);
[(Z)-(5-methyl-4-oxo-2-propan-2-ylcyclohexa-2,5-dien-1-ylidene)amino]4-methylbenzenesulfonate (Compound CID: 5941439);
[(2-methyl-4-oxo-5-propan-2-ylcyclohexa-2,5-dien-1-ylidene)amino]benzenesulfonate (Compound CID: 1909670);
[(5-methyl-4-oxo-2-propan-2-ylcyclohexa-2,5-dien-1-ylidene)amino]benzenesulfonate (Compound CID: 222113 8);
[(Z)-(2-methyl-4-oxo-5-propan-2-ylcyclohexa-2,5-dien-1-ylidene)amino]4-propan-2-ylbenzenesulfonate (Compound CID: 49787116);
[(E)-(5-cyclohexyl-2-methyl-4-oxocyclohexa-2;5-dien-1-ylidene)amino]3-chlorobenzenesulfonate (Compound CID: 49852493);
[(Z)-(5-cyclohexyl-2-methyl-4-oxocyclohexa-2;5-dien-1-ylidene)amino]3-methylbenzenesulfonate (Compound CID: 50898262);
[(Z)-(2-methyl-4-oxo-5-propan-2-ylcyclohexa-2,5-dien-1-ylidene)amino]3-methylbenzenesulfonate (Compound CID: 50898263);
[(Z)-(5-cyclohexyl-2-methyl-4-oxocyclohexa-2;5-dien-1-ylidene)amino]3-bromobenzenesulfonate (Compound CID: 50898264);
[(E)-(5-cyclohexyl-2-methyl-4-oxocyclohexa-2;5-dien-1-ylidene)amino]2-chlorobenzenesulfonate (Compound CID: 50898268);
[(Z)-(5-cyclohexyl-2-methyl-4-oxocyclohexa-2;5-dien-1-ylidene)amino]3-cyanobenzenesulfonate (Compound CID: 50898270);
(NZ)-N-[(4aR,8aS)-6,7-dimethyl-4-oxo-4a,5,8,8a-tetrahydronaphthalen-1-ylidene]benzenesulfonamide (Compound CID: 134879088);
[(4-oxo-3-phenylcyclohexa-2,5-dien- 1-ylidene) amino]benzenesulfonate (Compound CID: 50898267);
[(E)-(4-oxo-4-phenylbutan-2-ylidene)amino]benzenesulfonate (Compound CID: 88531954);
[(E)-(4-oxo-5-phenylpentan-2-ylidene)amino]benzenesulfonate (Compound CID: 88532017);
[(E)-(5-oxo-5-phenylpentan-2-ylidene)amino]benzenesulfonate (Compound CID: 88532051);
[(E)-(1-oxo-1-phenylpropan-2-ylidene)amino]benzenesulfonate (Compound CID: 88532204);
[(E)-(1-oxo-1-phenylpentan-3-ylidene)amino]benzenesulfonate (Compound CID: 88532276) ;
[(4-oxo-1-phenylpentan-2-ylidene)amino]benzenesulfonate (Compound CID: 88532489);
[(2,6-ditert-butyl-4,5-dioxocycloheX-2-en-1-ylidene)amino]benzenesulfonate (Compound CID: 123626753);
[(5-methyl-4-oxo-2-propan-2-ylcyclohexa-2,5-dien-1-ylidene)amino]4-methylbenzenesulfonate (Compound CID: 29065 67);
[(2-methyl-4-oxo-5-propan-2-ylcyclohexa-2,5-dien-1-ylidene)amino]4-methylbenzenesulfonate (Compound CID: 5245 197);
[(E)-(2-methyl-4-oxo-5-propan-2-ylcyclohexa-2,5-dien-1-ylidene)amino]4-methylbenzenesulfonate (Compound CID: 6311908);
[(Z)-(2-methyl-4-oxo-5-propan-2-ylcyclohexa-2,5-dien-1-ylidene)amino]4-methylbenzenesulfonate (Compound CID: 6536676); and
[(5-cyclohexyl-2-methyl-4-oxocyclohexa-2,5-dien-1-ylidene)amino]benzenesulfonate (Compound CID: 75249343).

In another aspect, the disclosure provides a kit for use in conjunction with any aspect of the disclosure described here, where the kit includes ML233, or analog(s) thereof. In certain embodiments of the kits described here, the kit further includes instructions for using ML233, or analog(s) thereof, to inhibit melanogenesis and/or treat melanoma.

### Definitions

All terms used herein are intended to have their ordinary meaning in the art unless otherwise provided. All concentrations are in terms of percentage by weight of the specified component relative to the entire weight of the composition, such as a topical or parenteral formulation, unless otherwise defined.

As used herein, “a” or “an” shall mean one or more. As used herein when used in conjunction with the word “comprising,” the words “a” or “an” mean one or more than one. As used herein “another” means at least a second or more.

As used herein, all ranges of numeric values include the endpoints and all possible values disclosed between the disclosed values. The exact values of all half-integral numeric values are also contemplated as specifically disclosed and as limits for all subsets of the disclosed range. For example, a range of from 0.1% to 3% specifically discloses a percentage of 0.1%, 1%, 1.5%, 2.0%, 2.5%, and 3%. Additionally, a range of 0.1 to 3% includes subsets of the original range including from 0.5% to 2.5%, from 1% to 3%, or from 0.1% to 2.5%. It will be understood that the sum of all weight % of individual components will not exceed 100%.

Throughout this description, various components can be identified having specific values or parameters, however, these items are provided as exemplary embodiments. Indeed, the exemplary embodiments do not limit the various aspects and concepts of the present disclosure as many comparable parameters, sizes, ranges, and/or values can be implemented. Unless otherwise specified, the terms “first,” “second,” and the like, “primary,” “secondary,” and the like, do not denote any order, quantity, or importance, but rather are used to distinguish one element from another.

By “administering” or “administration” as used herein, is meant any route of administration, such as but not limited to, topical, oral, and parenteral, for example, intradermal, transdermal, intralesional, subcutaneous, intramuscular, and intravenous.

By “agent” is meant a small compound, polypeptide or polynucleotide.

By “ameliorate” is meant decrease, suppress, attenuate, diminish, arrest, or stabilize the development or progression of a disease.

By “alteration” is meant a change (increase or decrease). In embodiments, the alteration is in the production of melanin. Methods for measuring melanin production are known in the art and described herein. As used herein, an alteration includes a 10% change in production levels, a 25% change, a 40% change, a 50% or an even greater change in melanin production levels.

By “analog” is meant here to mean a compound having a structure and/or functional similarity to that of another compound, such as ML233. “Analogs” include those having structural similarities, i.e., “structural analogs”; chemically different compounds having similar pharmacological properties, i.e., “functional analogs”; and those having similar chemical and pharmacological similarities, i.e., “direct analogs”.

By “brightening” is meant herein to brighten or lighten skin tone or skin pigmentation and/or evening out or making more uniform the skin tone or complexion of an individual by inhibiting melanogenesis, inhibiting tyrosinase, inhibiting melanin production or synthesis, and thereby treating pigmentation diseases or conditions, such as but not limited to, undesirable hyperpigmentation or hypopigmentation, Addison's disease, melasma, solar lentigo, or vitiligo.

By “consists essentially” it is meant that the ingredients include only the listed components along with the normal impurities present in commercial materials and with any other additives present at levels which do not affect the operation of the disclosure, for instance at levels less than 5% by weight or less than 1% or even 0.5% by weight.

By “contacting” as used herein, is meant that the compound described here (e.g., ML233, or analog(s) thereof) or composition comprising the compound, or analog(s) thereof, either directly or indirectly, is administered to an affected cell or a cell in need of treatment (e.g., a cell, a tissue, a subject).

By “disease” is meant any condition or disorder that damages or interferes with the normal function of a cell, tissue, or organ. Compounds described herein, or analog(s) thereof, are useful for the treatment of diseases, including for example, melanomas, hyperpigmentation, mixed hyper-/hypopigmentation disorders, melasma, post-inflammatory hyperpigmentation, Addison's disease, solar lentigo, vitiligo, and Addison's disease.

By “disease associated with melanin production” is meant a disease characterized by undesirable levels of melanin production. In some embodiments, the disease (e.g., Addison's disease, hyperpigmentation, melasma, solar lentigo) is associated with an undesirable increase in pigmentation. In other embodiments, the disease (vitiligo, hypopigmentation) is associated with an undesirable decrease in pigmentation, which decrease might be made less noticeable by reducing the level of pigmentation in surrounding cells and/or tissues.

“Detect” refers to identifying the presence, absence or amount of the analyte to be detected. In come embodiments, the analyte is melanin, or a precursor or derivative thereof.

The term “effective amount” or “therapeutically effective amount” of an agent is meant the amount of an agent (e.g., a compound described herein (e.g., ML233, or analog(s) thereof)) required to ameliorate the symptoms of a disease relative to an untreated patient. The effective amount of active compound(s), or analog(s) thereof, used to practice the present disclosure for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount sufficient to reduce or decrease pigmentation, tyrosinase activity, or melanin production or melanogenesis, or sufficient to treat or ameliorate skin diseases or conditions, such as but not limited to, skin cancers, such as melanoma, vitiligo, melasma, hyperpigmentation, hypopigmentation, hypomelanic macules, and the like. Agents described herein include compounds having the structure of Formula (I), or analog(s) thereof, or salts thereof, or any of those identified in the present disclosure. In some embodiments, the compounds are administered in an effective amount for the treatment or prophylaxis of a disease disorder or condition. In another embodiment, in the context of administering an agent that is an melanogenesis inhibiting agent, an effective amount of an agent is, for example, an amount sufficient to achieve alleviation or amelioration or prevention or prophylaxis of one or more symptoms or conditions; diminishment of extent of disease, disorder, or condition associated with melanoma; stabilized (i.e., not worsening) state of disease, disorder, or condition; preventing spread of disease, disorder, or condition; delay or slowing the progress of the disease, disorder, or condition; amelioration or palliation of the disease, disorder, or condition (e.g., those associated with infection); and remission (whether partial or total), whether detectable or undetectable, as compared to the response obtained without administration of the agent. In some embodiments, the compound described herein, or analog(s) thereof, slows the rate of spread or decreases the size of a melanoma in a host subject or infected medium.

The term “ML233” refers to an apelin receptor agonist (E)-2-cyclohexyl-5-methyl-4-(phenylsulfonyloxyimino)cyclohexa-2,5-dienone (IUPAC NAME: [(E)-(5-cyclohexyl-2-methyl-4-oxocyclohexa-2,5-dien-1-ylidene)amino]benzenesulfonate), having formula: C₁₉H₂₁NO₄S, and CAS number: 2080311-92-6, PubChem Compound CID: 60138111, PubChem SID: 381994294, External ID: AA01EP2Q; or an enantiomer, diastereomer, or mixture of enantiomers and/or diastereomers (e.g., racemic mixture) of the compound of formula (I). ML233 is known in the art and described, for example, in Yang et al (2015) Trends Pharmacol Sci. 36 560 PMID: 26143239; and Khan et al (2011) Probe Reports from the NIH Molecular Libraries Program PMID: 22834038. An exemplary structure for ML233 is provided below:

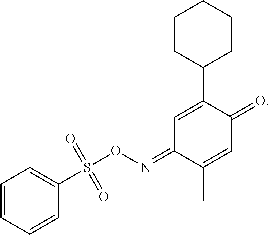 [SMILES: CC1=CC(=O)C(C2CCCCC2)=C/C1=N\OS(=O)(=O)c1ccccc1]

Non-limiting examples of ML233 analogs include those below (structures, IUPAC names, Compound CID, and/or PubChem SID or External ID):

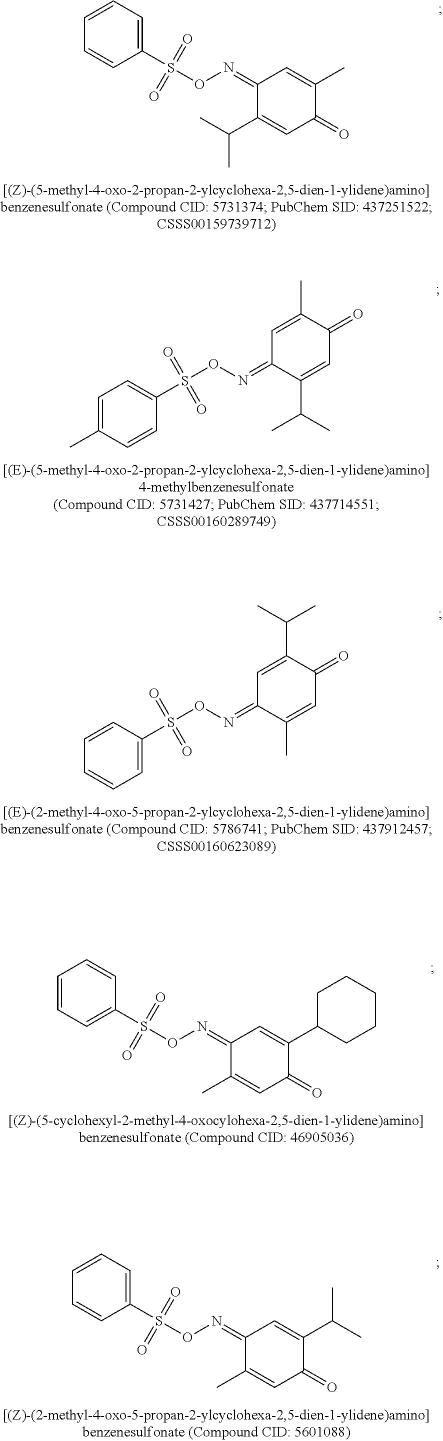 [SMILES: CC1=C/C(=N/OS(=O)(=O)c2ccccc2)C(C(C)C)=CC1=O ; CC1=C/C(=N\OS(=O)(=O)c2ccc(C)cc2)C(C(C)C)=CC1=O ; CC1=CC(=O)C(C(C)C)=C/C1=N\OS(=O)(=O)c1ccccc1 ; CC1=CC(=O)C(C2CCCCC2)=C/C1=N/OS(=O)(=O)c1ccccc1 ; CC1=CC(=O)C(C(C)C)=C/C1=N/OS(=O)(=O)c1ccccc1]

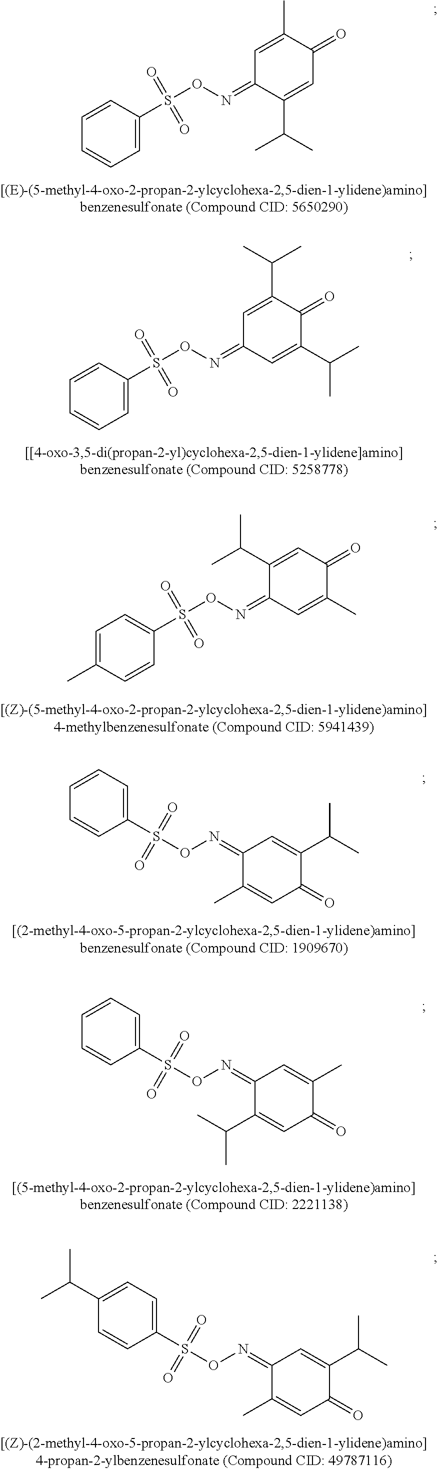

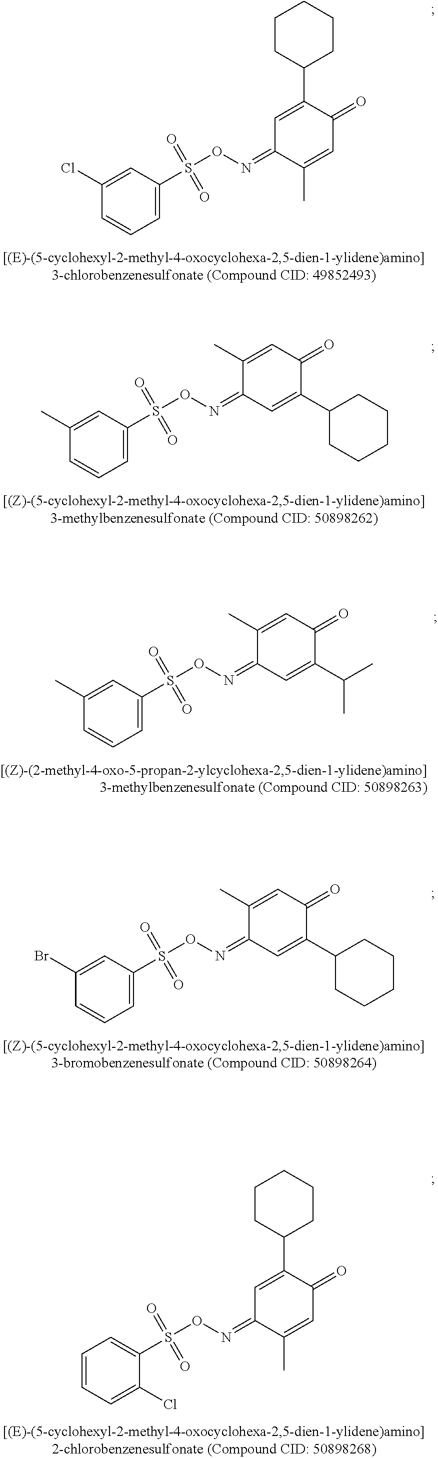 [SMILES: CC1=CC(=O)C(C2CCCCC2)=C/C1=N\OS(=O)(=O)c1cccc(Cl)c1 ; CC1=CC(=O)C(C2CCCCC2)=C/C1=N/OS(=O)(=O)c1cccc(C)c1 ; CC1=CC(=O)C(C(C)C)=C/C1=N/OS(=O)(=O)c1cccc(C)c1 ; CC1=CC(=O)C(C2CCCCC2)=C/C1=N/OS(=O)(=O)c1cccc(Br)c1 ; CC1=CC(=O)C(C2CCCCC2)=C/C1=N\OS(=O)(=O)c1ccccc1Cl]

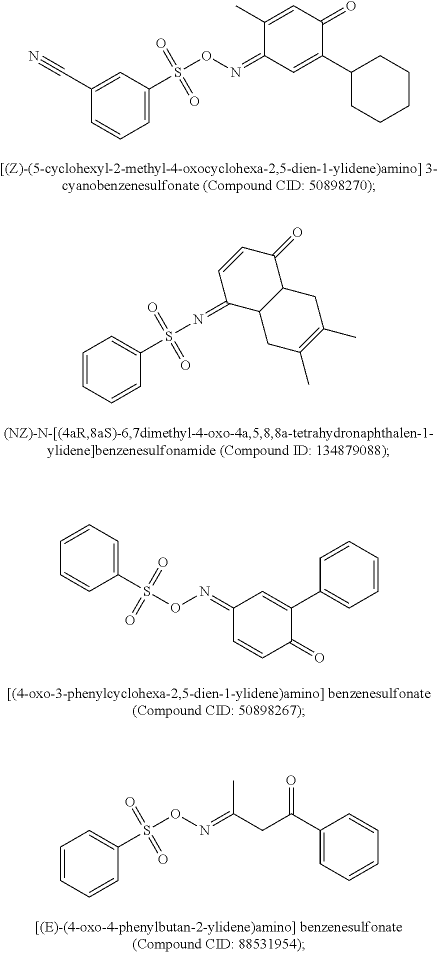 [SMILES: CC1=CC(=O)C(C2CCCCC2)=C/C1=N/OS(=O)(=O)c1cccc(C#N)c1 ; CC1=C(C)CC2C(=NS(=O)(=O)c3ccccc3)C=CC(=O)C2C1 ; O=C1C=C/C(=N\OS(=O)(=O)c2ccccc2)C=C1c1ccccc1 ; C/C(CC(=O)c1ccccc1)=N\OS(=O)(=O)c1ccccc1]

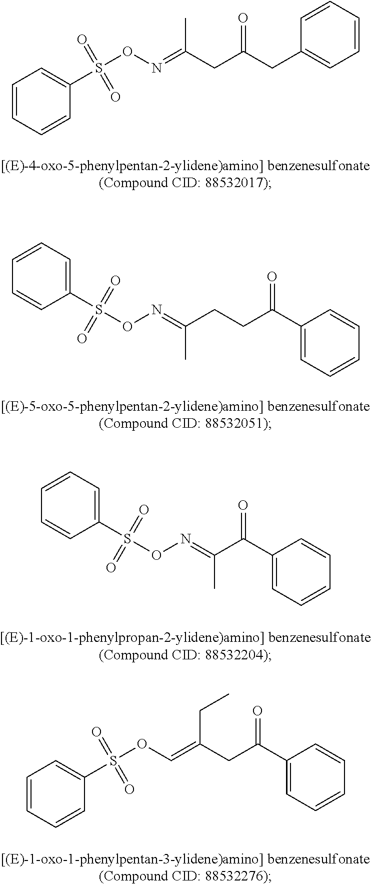 [SMILES: C/C(CC(=O)Cc1ccccc1)=N\OS(=O)(=O)c1ccccc1 ; C/C(CCC(=O)c1ccccc1)=N\OS(=O)(=O)c1ccccc1 ; C/C(=N\OS(=O)(=O)c1ccccc1)C(=O)c1ccccc1 ; CC/C(=C\OS(=O)(=O)c1ccccc1)CC(=O)c1ccccc1]

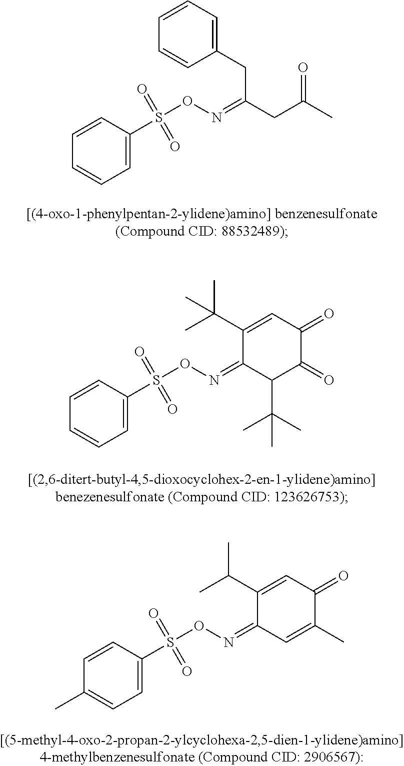

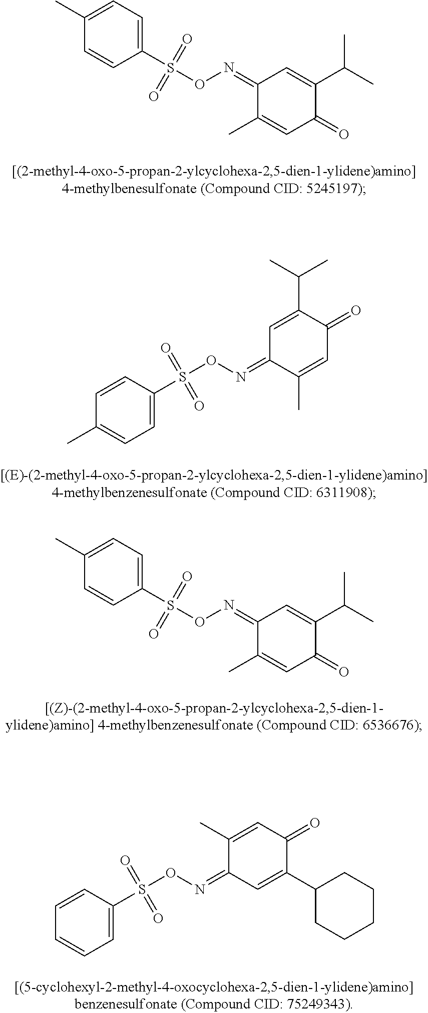

The term “pharmaceutical composition,” as used herein, represents a composition containing a compound described herein, or analog(s) thereof, formulated with a pharmaceutically acceptable excipient. In some embodiments, the pharmaceutical composition is manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gel cap); for topical administration (e.g., as a cream, gel, lotion, or ointment); for intravenous administration (e.g., as a sterile solution free of particulate emboli and in a solvent system suitable for intravenous use); or in any other formulation described herein (see below). In an embodiment, the pharmaceutical composition comprises ML233, or analog(s) thereof, or a salt or derivative thereof.

As used herein, the phrase “pharmaceutically acceptable” generally safe for ingestion or contact with biologic tissues at the levels employed. Pharmaceutically acceptable is used interchangeably with physiologically compatible. It will be understood that the pharmaceutical compositions of the disclosure include nutraceutical compositions (e.g., dietary supplements) unless otherwise specified. It will also be understood that the disclosure also includes topical compositions for use on a subject's skin. It will also be understood that the disclosure includes non-toxic compositions for ingestion, cutaneous application, or intravenous injection.

By “reduces” is meant a negative alteration of at least 10%, 25%, 50%, 75%, or 100%.

By “reference” is meant a standard or control condition.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

By “subject” is meant a mammal, including, but not limited to, a human or non-human mammal, such as a bovine, equine, canine, ovine, or feline. Typical subjects include any animal (e.g., mammals such as mice, rats, rabbits, non-human primates, and humans). A subject in need thereof is typically a subject for whom it is desirable to treat a disease, disorder, or condition as described herein. For example, a subject in need thereof can seek or be in need of treatment, require treatment, be receiving treatment, can be receiving treatment in the future, or a human or animal that is under care by a trained professional for a particular disease, disorder, or condition.

The term “substituent” refers to a group “substituted” on a hydrocarbon, e.g., an alkyl, at any atom of that group, replacing one or more atoms therein (e.g., the point of substitution) including hydrogen atoms or carbon atoms. In some aspects, the substituent(s) on a group are independently any one single, or any combination of two or more of the permissible atoms or groups of atoms delineated for that substituent. In another aspect, a substituent can itself be substituted with any one of the substituents described herein. Substituents can be located pendant to the hydrocarbon chain.

In addition, the phrase “substituted with a[n],” as used herein, means the specified group can be substituted with one or more of any or all of the named substituents. For example, where a group, such as an alkyl or heteroaryl group, is “substituted with an unsubstituted C1-C20 alkyl, or unsubstituted 2 to 20 membered heteroalkyl,” the group can contain one or more unsubstituted C1-C20 alkyls, and/or one or more unsubstituted 2 to 20 membered heteroalkyls. Moreover, where a moiety is substituted with an R substituent, the group can be referred to as “R-substituted.” Where a moiety is R-substituted, the moiety is substituted with at least one R substituent and each R substituent is optionally different (e.g., R can be independently selected at each occurrence from C1-C10 alkyl optionally comprising one or more points of substitution).

As used herein, the terms “treat,” treating,” “treatment,” and the like refer to reducing or ameliorating a disease, disorder, conditions, and/or symptoms associated therewith. It will be appreciated that, although not precluded, treating a disease, disorder, or condition does not require that the disorder, condition or symptoms associated therewith be completely eliminated. For example, ML233, or analog(s) thereof, or salts thereof, or compositions comprising such compounds or analog(s) thereof, can be administered to a subject in need thereof, to treat a disease or condition associated with the production of melanin in the subject, such as but not limited to, melanogenesis dysfunction or dysregulation, vitiligo, melasma, hyperpigmentation, hypopigmentation, hypomelanic macules, Addison's disease, solar lentigo, senile lentigo, and skin cancers, such as melanoma.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1A provides a series of micrographs showing that ML233 treatment inhibited pigmentation in zebrafish embryos. Embryos were exposed to ML233 4 hours post fertilization (hpf), and the experiment was concluded at 48 hpf. ML233 was dissolved in DMSO as a carrier. Pigmentation was analyzed at 2.5 micromolar ML233, 5 micromolar ML233, and 10 micromolar ML233.

FIG. 1B provides a bar chart showing a change in the percentage of melanin quantity in response to ML233 in zebrafish embryos. Embryos were exposed to ML233 4 hours post fertilization (hpf), and the experiment was concluded at 48 hpf. DMSO was used as a negative control. Phenylthiourea (PTU), which is a potent tyrosinase inhibitor, was used as a positive control. Each condition has been quantified with biological triplicates. Error bars represent SD. *p<0.05, **p<0.001, ***p<0.0005, n.s, not significant; determined by t-test, two-tailed.

FIG. 2A shows micrographs of zebrafish embryos treated for six hours with ML233 at 5 micromolar, 10 micromolar, and 15 micromolar. DMSO was used as a negative control.

FIG. 2B provides micrographs of zebrafish embryos incubated for 24 hours with ML233 at 5 micromolar, 10 micromolar, and 15 micromolar. DMSO was used as a negative control.

FIG. 3A includes a bar chart showing the effects of ML233 treatment in vivo on percentage of tyrosinase activity. Zebrafish embryos were treated with ML233 concentration levels between 0.5 micromolar and 15 micromolar. DMSO was used as a negative control, and PTU was used as a positive control. Each condition has been quantified with biological triplicates. Error bars represent SD. *p<0.05, **p<0.001, ***p<0.0005, n.s, not significant; determined by t-test, two-tailed.

FIG. 3B shows a bar chart showing the effects of ML233 treatment in vitro on the percentage of tyrosinase activity of a sample treated with 20 or 30 micromolar ML233. DMSO was used as a negative control, and Kojic acid was used as a positive control. Each condition has been quantified with biological duplicates.

FIG. 3C shows a bar chart showing the effects of ML233 treatment in vitro on the percentage of tyrosinase activity of a sample treated with 20 micromolar ML233. DMSO was used as a negative control, and Kojic acid (100 micromolar) was used as a positive control. (n=4). Error bars represent SD. Statistical significance is determined by one-way analysis of variance (ANOVA).

FIG. 4A provides a table depicting the percentage of viable zebrafish embryos under different experimental conditions, including non-treated embryos, DMSO treated embryos, 2.5 micromolar ML233, 5 micromolar ML233, 10 micromolar ML233, and 20 micromolar ML233, where the non-treated and DMSO groups are of size n=25 and the ML233 groups are of size n=20. The number of dead embryos at one day post fertilization (dpf), two dpf, three dpf, and four dpf are shown, as well as the number of viable embryos, and the percentage of viability.

FIG. 4B provides a bar chart showing the percentage of viable zebrafish embryos at one dpf to four dpf under the following experimental conditions: 2.5 micromolar ML233, five micromolar ML233, 10 micromolar ML233, and 20 micromolar ML233. A non-treated group and a DMSO group were used as negative controls.

FIG. 5A provides a graph showing the effect of ML233 on B16-F10 proliferation. Cell viability of control percentage is quantified as a function of ML233 concentration.

FIG. 5B provides a graph showing the effect of cisplatin on B16-F10 proliferation. Cell viability of control percentage is quantified as a function of cisplatin concentration.

FIG. 6A provides a bar chart depicting changes in the optical density (OD) value of melanin in response to treatment with ML233. DMSO was used as a negative control. IBMX was used to stimulate melanin production in murine B16-F10 melanoma cells. Each condition has been quantified with biological triplicates. Error bars represent SD. *p<0.05, **p<0.01, ***p<0.001, ****p<0.0001, n.s, not significant; determined by t-test, two-tailed.

FIG. 6B provides a bar chart depicting ratios with the total quantity of protein (OD value 562) that was calculated. ML233 reduced melanogenesis in mammalian melanoma cells at all dosages. P-values are indicated in the bar charts.

FIG. 7 shows a two-dimensional representation of a predicted binding site for ML233 in the tyrosinase protein, whereby ML233 stably comes into contact with the tyrosinase protein. Computational modeling and molecular docking were used to predict the binding of ML233 with the human tyrosinase protein. ML233 potentially bounds in the ligand binding pocket of the human tyrosinase protein.

FIG. 8 provides a two-dimensional representation of a predicted binding site for ML233 in the tyrosinase protein, whereby ML233 stably comes into contact with the tyrosinase protein. Computational modeling and molecular docking were used to predict the binding of ML233 with the human tyrosinase protein. ML233 potentially interacts with stability in the ligand binding pocket of the human tyrosinase protein.

FIG. 9A provides a western blot analysis of tyrosinase expression after ML233 treatment at 15 micromolar. DMSO was used as a negative control. CRISPR directed against the tyrosinase gene was used as a control for the specificity of the antibody used in this analysis. Phenylthiourea (PTU) was used as a positive control.

FIG. 9B provides a quantification by densitometry analysis of tyrosinase expression after ML233 treatment at 15 micromolar. DMSO was used as a negative control. CRISPR directed against the tyrosinase gene was used as a control for the specificity of the antibody used in this analysis. Phenylthiourea (PTU) was used as a positive control.

FIGS. 10A-10F shows that ML233-dependent regulation of TYR function is not mediated at the transcriptional level. FIG. 10A provides the analysis of tyr, dct and mitfa mRNA expression by RT-qPCR in DMSO, ML233 or PTU treated (between 24 and 48 hpf) zebrafish embryos at 48 hpf (n=3; ≥40 embryos/sample). FIG. 10B provides the analysis of mitfa and tyr mRNA expression by in situ hybridization in ML233- or PTU-treated (between 24 and 48 hpf) embryos at 48 hpf. FIGS. 10C-10F show the graphical representation of bulk RNAseq analysis (n=4; ≥20 embryos/sample) and variation in gene expression profiles in control vs apelin receptors KO mutant (FIG. 10C), after 3 hours of non-treated vs ML233-treated in control (FIG. 10D), after 3 hours of non-treated vs ML233-treated in apelin receptors KO mutant (FIG. 10E), and after 24 hours of non-treated vs ML233-treated in apelin receptors KO mutant (FIG. 10F). Scale bars: 100 m. Error bars represent SD. Significance was determined by t-test, two-tailed, unpaired.

FIGS. 11A-11D demonstrate that ML233-dependent regulation of TYR function is not mediated through protein degradation. FIG. 11A demonstrates expression of tyrosinase protein analyzed by Western blot in murine (B16F10) or human (A375) melanoma cells (n=3). FIG. 11B shows expression of tyrosinase protein analyzed by Western blot in murine (B16F10) melanoma cells after DMSO or ML233 treatment (n=3). FIG. 11C shows that tyrosinase protein expression quantified and normalized by expression of the beta-actin protein after DMSO or ML233 treatment. FIG. 11D shows representative pictures of melanin expression in murine (B16F10) melanoma cells after DMSO or ML233 treatment. Error bars represent SD. Significance was determined by t-test, two-tailed, unpaired.

FIGS. 12A-12B demonstrated that ML233 inhibited melanoma proliferation in mammals. Cell proliferation in patient-derived xenograft organoid (PDXO) cellular models was quantified in ME1154B (FIG. 12A) and ME2319B (FIG. 12B) human melanoma lines after staurosporine (STS, positive control) or ML233 treatment (n=3). Error bars represent SD. Statistical significance was determined by one-way analysis of variance (ANOVA).

FIG. 13 shows the quantification of the binding free energy during TYR and ML233 interaction.

### DETAILED DESCRIPTION

The present disclosure provides melanin-inhibiting compounds for disease treatment or skin brightening, pharmaceutical compositions comprising such melanin-inhibiting compounds, and methods of use thereof.

The disclosure is based, at least in part, on the discovery of tyrosinase activity and pigmentation inhibition via (E)-2-cyclohexyl-5-methyl-4-(phenylsulfonyloxyimino)cyclohexa-2,5-dienone (ML233), or analog(s) thereof. This effect is reversible and is likely linked to the regulation of tyrosinase protein activity. The present disclosure provides experimental validation in the zebrafish model organism, in vivo, that ML233 reduces melanin production and skin pigmentation without affecting the survival of melanocytes, and melanogenesis is inhibited by downregulation of tyrosinase activity. Additionally, ML233 was demonstrated to occupy or contact tyrosinase binding sites, thereby preventing the hydroxylation of L-tyrosine into L-3,4-dihydroxyphenylalanine (L-DOPA) and melanogenesis from occurring.

In some embodiments, the compound having the structure of formula (I):

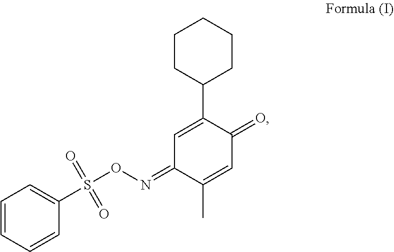 [SMILES: CC1=CC(=O)C(C2CCCCC2)=C/C1=N\OS(=O)(=O)c1ccccc1]

also known as (E)-2-cyclohexyl-5-methyl-4-(phenylsulfonyloxyimino)cyclohexa-2,5-dienone (ML233) (IUPAC NAME: [(E)-(5-cyclohexyl-2-methyl-4-oxocyclohexa-2,5-dien-1-ylidene)amino]benzenesulfonate; CAS number: 2080311-92-6, PubChem Compound CID: 60138111, PubChem SID: 381994294, External ID: AA01EP2Q), (ML233), or analog(s) thereof, or salts thereof, or an enantiomer, a diastereomer, or a mixture of enantiomers and/or diastereomers (e.g., racemic mixture) thereof, or a pharmaceutically acceptable salt thereof, inhibits melanogenesis, tyrosinase activity, and pigmentation. ML233 or analog(s) thereof can inhibit tyrosinase (function or activity) by binding or occupying the ligand-binding pocket of the tyrosinase protein. In some aspects, ML233 or analog(s) thereof binds one or more asparagine N-glycosylation sites of tyrosinase (i.e., Asn 86, Asn 111, Asn 230, Asn 290, and/or Asn 371). Further aspects provide for ML233 or analog(s) thereof that bind one or more copper ion binding sites. Additional aspects include those where ML233 or analog(s) thereof bind tyrosinase at one or more positions: His180, Glu345, Ser360, His367, Ile368, and Val377. In some aspects, ML233 or analog(s) thereof form hydrophobic interactions with one or more of: Ile368, Phe207, Phe386, and Val377 of tyrosinase, and polar van der Waals interactions with one or more of: His180, His202 His367, His390, Ser380, and G1n376 of tyrosinase. ML233 or analog(s) thereof bind or occupy any one or more of these binding sites in an amount sufficient to inhibit tyrosinase (i.e., function or regulation) or its activity, inhibit melanogenesis, inhibit melanin production, even out skin pigmentation or skin tone, brighten skin pigmentation or skin tone, inhibit cell proliferation of melanomas, ameliorate or treat skin cancer (e.g., melanoma). Non-limiting examples of ML233 analogs include those below (structures, IUPAC names, Compound CID, and/or PubChem SID or External ID):

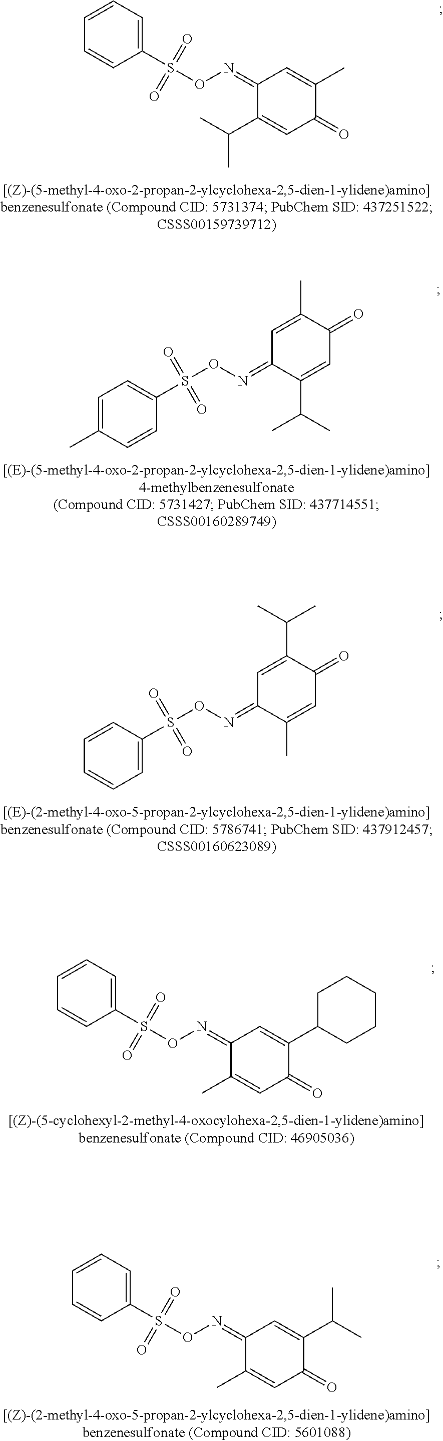 [SMILES: CC1=C/C(=N/OS(=O)(=O)c2ccccc2)C(C(C)C)=CC1=O ; CC1=C/C(=N\OS(=O)(=O)c2ccc(C)cc2)C(C(C)C)=CC1=O ; CC1=CC(=O)C(C(C)C)=C/C1=N\OS(=O)(=O)c1ccccc1 ; CC1=CC(=O)C(C2CCCCC2)=C/C1=N/OS(=O)(=O)c1ccccc1 ; CC1=CC(=O)C(C(C)C)=C/C1=N/OS(=O)(=O)c1ccccc1]

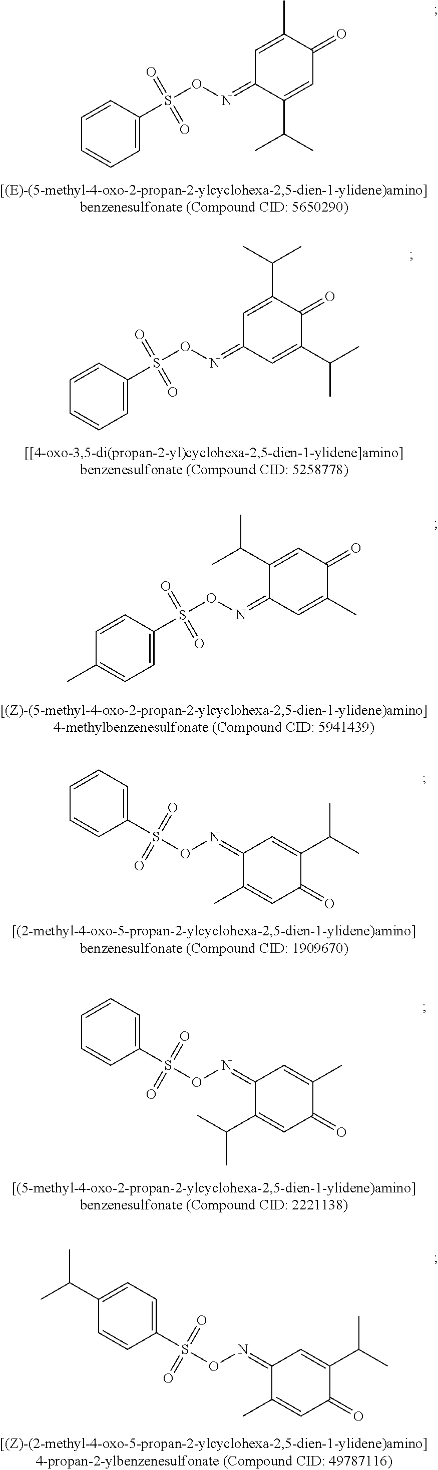

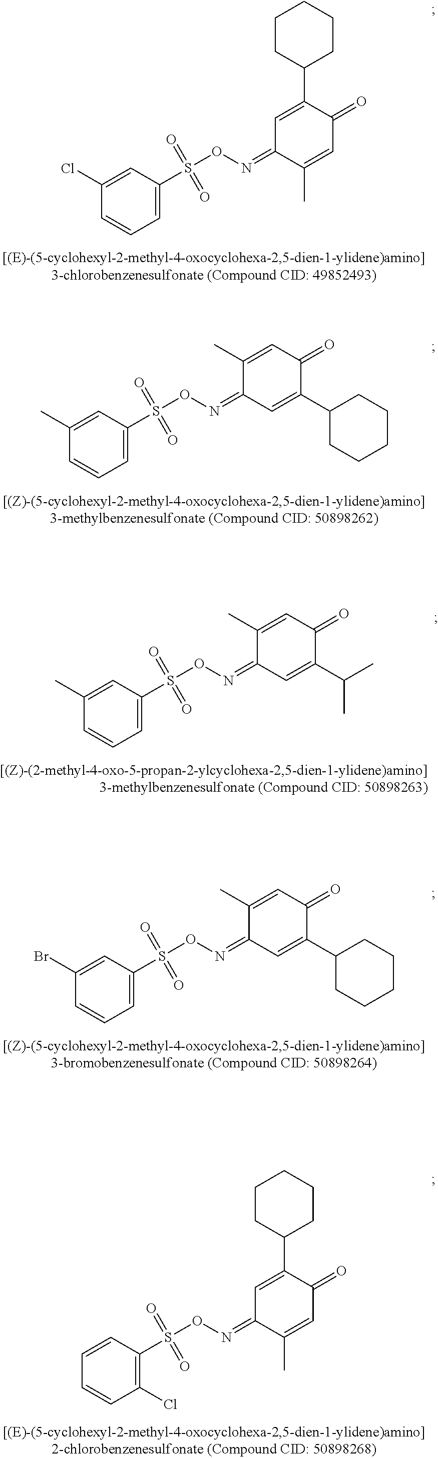 [SMILES: CC1=CC(=O)C(C2CCCCC2)=C/C1=N\OS(=O)(=O)c1cccc(Cl)c1 ; CC1=CC(=O)C(C2CCCCC2)=C/C1=N/OS(=O)(=O)c1cccc(C)c1 ; CC1=CC(=O)C(C(C)C)=C/C1=N/OS(=O)(=O)c1cccc(C)c1 ; CC1=CC(=O)C(C2CCCCC2)=C/C1=N/OS(=O)(=O)c1cccc(Br)c1 ; CC1=CC(=O)C(C2CCCCC2)=C/C1=N\OS(=O)(=O)c1ccccc1Cl]

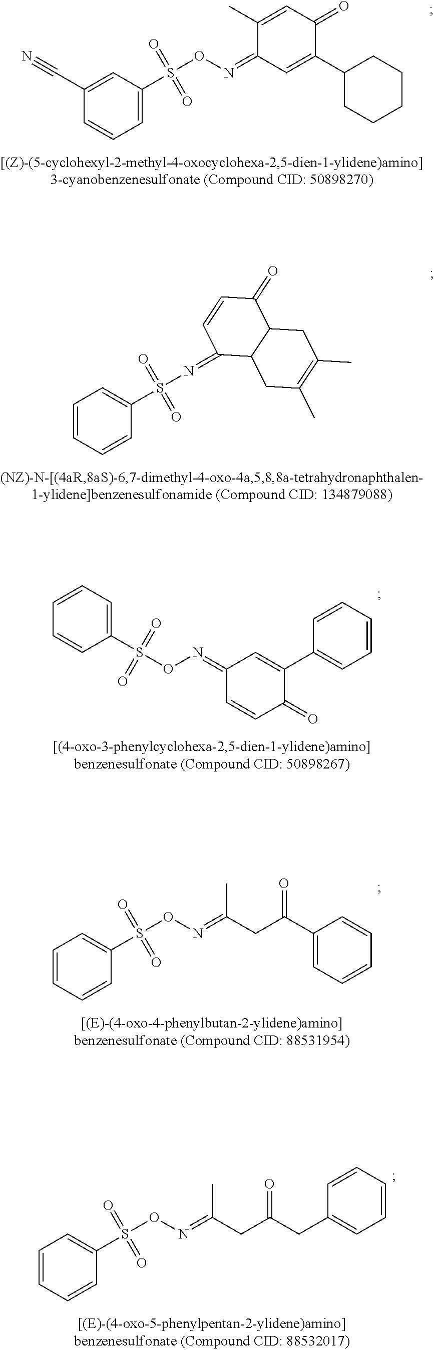 [SMILES: CC1=CC(=O)C(C2CCCCC2)=C/C1=N/OS(=O)(=O)c1cccc(C#N)c1 ; CC1=C(C)CC2C(=NS(=O)(=O)c3ccccc3)C=CC(=O)C2C1 ; O=C1C=C/C(=N\OS(=O)(=O)c2ccccc2)C=C1c1ccccc1 ; C/C(CC(=O)c1ccccc1)=N\OS(=O)(=O)c1ccccc1 ; C/C(CC(=O)Cc1ccccc1)=N\OS(=O)(=O)c1ccccc1]

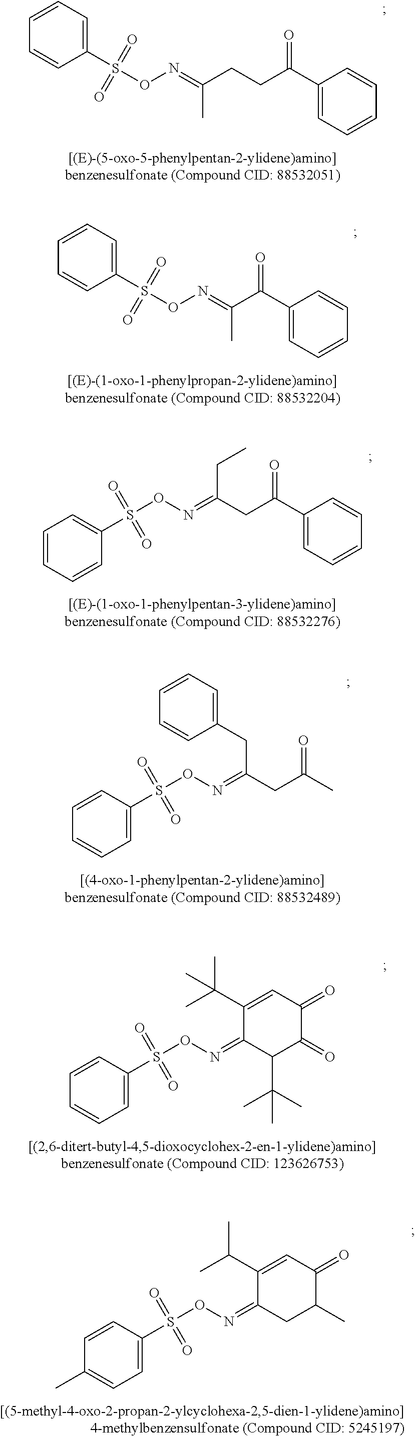

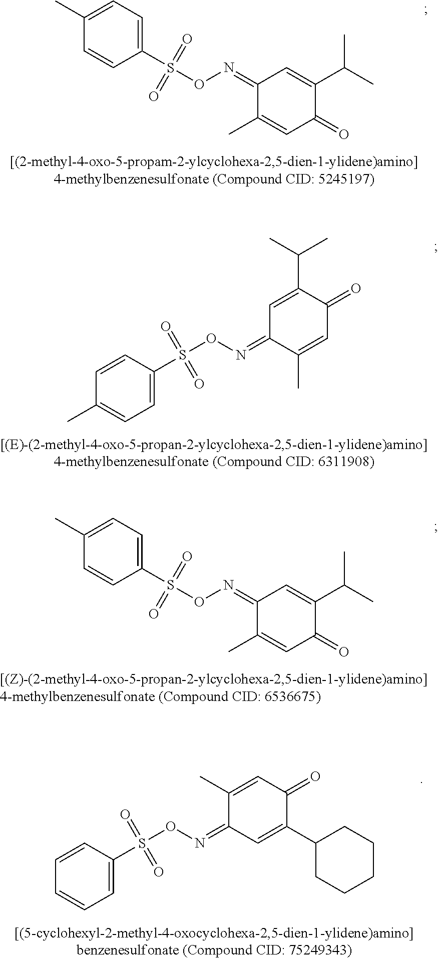

In some embodiments, the compound of Formula (I), or analog(s) thereof, can have one or more asymmetric carbon atoms and can exist in the form of optically pure enantiomers, mixtures of enantiomers such as racemates, optically pure diastereoisomers, mixtures of diastereoisomers, diastereoisomeric racemates or mixtures of diastereoisomeric racemates. The optically active forms can be obtained for example by resolution of the racemates, by asymmetric synthesis or asymmetric chromatography (chromatography with a chiral adsorbent or eluant). That is, certain of the disclosed compounds can exist in various stereoisomeric forms including stereoisomers, enantiomers, diastereomers, or racemates (i.e., the compound exists as a mixture containing two enantiomers and does not rotate polarized light). Enantiomers of a compound can be prepared, for example, by separating an enantiomer from a racemate using one or more well-known techniques and methods, such as chiral chromatography and separation methods based thereon. The appropriate technique and/or method for separating an enantiomer of a compound described herein from a racemic mixture can be readily determined by those of skill in the art.

The compound or analog(s) thereof provided herein can also be present as geometric isomer which differ in the orientation of substituent atoms (e.g., to a carbon-carbon double bond, to a cycloalkyl ring, to a bridged bicyclic system). Atoms (other than H) on each side of a carbon-carbon double bond can be in an E (substituents are on opposite sides of the carbon-carbon double bond) or Z (substituents are oriented on the same side) configuration. “R,” “S,” “S*,” “R*,” “E,” “Z,” “cis,” and “trans,” indicate configurations relative to the core molecule and can be used to indicate the geometric configuration of the presently disclosed compounds. Certain of the disclosed compounds, or analog(s) thereof, can exist in atropisomeric forms. Atropisomers are stereoisomers resulting from hindered rotation about single bonds where the steric strain barrier to rotation is high enough to allow for the isolation of the conformers.

The compounds disclosed herein, or analog(s) thereof, can be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods. When the stereochemistry of a disclosed compound is named or depicted by structure, the named or depicted stereoisomer can be typically more than 50% (e.g., at least 55%, 60%, 70%, 80%, 90%, 99%, or 99.9%) by weight (or mole fraction) relative to the other stereoisomers. When a single enantiomer is named or depicted by structure, the depicted or named enantiomer is more than 50% (e.g., at least 55%, 60%, 70%, 80%, 90%, 99%, or 99.9%) by weight (or mole fraction) optically pure. When a single diastereomer is named or depicted by structure, the depicted or named diastereomer is more than 50% (e.g., at least 55%, 60%, 70%, 80%, 90%, 99%, or 99.9%) by weight (or mole fraction) pure. Percent optical purity is the ratio of the weight of the enantiomer or over the weight of the enantiomer plus the weight of its optical isomer. Diastereomeric purity by weight is the ratio of the weight of one diastereomer or over the weight of all the diastereomers. Percent purity by mole fraction is the ratio of the moles of the enantiomer or over the moles of the enantiomer plus the moles of its optical isomer. Similarly, percent purity by moles fraction is the ratio of the moles of the diastereomer or over the moles of the diastereomer plus the moles of its isomer. When a disclosed compound, or analog(s) thereof, is named or depicted by structure without indicating the stereochemistry, and the compound has at least one chiral center, it is to be understood that the name or structure encompasses either enantiomer of the compound free from the corresponding optical isomer, a racemic mixture of the compound or mixtures enriched in one enantiomer relative to its corresponding optical isomer. When a disclosed compound, or analog(s) thereof, is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s) or mixtures of diastereomers in which one or more diastereomer is enriched relative to the other diastereomers. The disclosure embraces all of these forms.

Solvates of the compounds described herein, or analog(s) thereof, can form the aggregate of the compound or an ion of the compound with one or more solvents. Such solvents cannot interfere with the biological activity of the solute. Examples of suitable solvents include, but are not limited to, water, methanol (MeOH), ethanol (EtOH), and acetic acid (AcOH). Solvates where water is the solvent molecule are typically referred to as hydrates. Hydrates include compositions containing stoichiometric amounts of water, as well as compositions containing variable amounts of water.

In some embodiments, the compound of Formula (I) described here, or analog(s) thereof, can be present as a pharmaceutically acceptable salt. Typically, salts are composed of a related number of cations and anions (at least one of which is formed from the compounds described herein, or analog(s) thereof) coupled together (e.g., the pairs can be bonded ionically) such that the salt is electrically neutral. Pharmaceutically acceptable salts can retain or have similar activity to the parent compound, or analog(s) thereof, (e.g., an ED₅₀ within 10%) and have a toxicity profile within a range that affords utility in pharmaceutical compositions. For example, pharmaceutically acceptable salts can be suitable for use in contact with the tissues of humans and animals without undue toxicity, irritation, allergic response and are commensurate with a reasonable benefit/risk ratio. Pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. Salts can be prepared from pharmaceutically acceptable non-toxic acids and bases including inorganic and organic acids and bases. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, dichloroacetate, digluconate, dodecylsulfate, ethanesulfonate, formate, fumarate, glucoheptonate, glutamate, glycerophosphate, hemisulfate, heptonate, hexanoate, hippurate, hydrobromide, hydrochloride, hydroiodide, 2-hydroxy-ethanesulfonate, isethionate, lactobionate, lactate, laurate, lauryl sulfate, malate, maleate, malonate, mandelate, methanesulfonate, mucate, 2-naphthalenesulfonate, nicotinate, nitrate, oleate, oxalate, palmitate, pamoate, pantothenate, pectinate, persulfate, 3-phenylpropionate, phosphate, picrate, pivalate, propionate, stearate, succinate, sulfate, tartrate, thiocyanate, toluenesulfonate, undecanoate, and valerate salts. Representative basic salts include alkali or alkaline earth metal salts include sodium, lithium, potassium, calcium, and magnesium, aluminum salts, as well as nontoxic ammonium, quaternary ammonium, and amine cations, including, but not limited to ammonium, tetramethylammonium, tetraethylammonium, methylamine, dimethylamine, trimethylamine, triethylamine, caffeine, and ethylamine.

Pharmaceutically acceptable acid addition salts of the disclosure can be formed by the reaction of a compound of the disclosure, or analog(s) thereof, with an equimolar or excess amount of acid. Alternatively, hemi-salts can be formed by the reaction of a compound of the disclosure, or analog(s) thereof, with the desired acid in a 2:1 ratio, compound to acid. The reactants are generally combined in a mutual solvent such as diethyl ether, tetrahydrofuran, methanol, ethanol, iso-propanol, benzene, or the like. The salts normally precipitate out of solution within, e.g., one hour to ten days and can be isolated by filtration or other conventional methods.

The compounds of the present disclosure, or analog(s) thereof, include the compounds themselves, as well as their salts, if applicable. A salt, for example, can be formed between an anion and a positively charged substituent (e.g., amino) on a compound described herein, or analog(s) thereof. Suitable anions include chloride, bromide, iodide, sulfate, nitrate, phosphate, citrate, methanesulfonate, trifluoroacetate, and acetate. Likewise, a salt can also be formed between a cation and a negatively charged substituent (e.g., carboxylate) on a compound described herein, or analog(s) thereof. Suitable cations include sodium ion, potassium ion, magnesium ion, calcium ion, and an ammonium cation such as tetramethylammonium ion.

### Tyrosinase Inhibitors

Tyrosinase catalyzes the rate-limiting step of melanin synthesis within melanocytes, making it a key part of melanogenesis. Tyrosinase inhibitors can reduce synthesis or presence of melanin in order to reduce skin pigmentation, or inhibit the growth, proliferation, and survival of melanoma cells. Tyrosinase inhibitors, such as the compound of Formula (I), or analog(s) thereof, or an enantiomer, a diastereomer, or a mixture of enantiomers and/or diastereomers (e.g., racemic mixture) thereof, or a pharmaceutically acceptable salt thereof, therefore, can be used to inhibit melanogenesis and/or reduce melanin in a subject. Such tyrosinase inhibitors comprising ML233, or analog(s) thereof, inhibit tyrosinase (i.e., function or regulation) or its activity, inhibit melanogenesis, inhibit melanin production, even out skin pigmentation or skin tone, brighten skin pigmentation or skin tone, inhibit cell proliferation of melanomas, ameliorate or treat skin cancer (e.g., melanoma). In some embodiments, compounds of the disclosure, or analog(s) thereof, for example, Formula (I), or analog(s) thereof, or compositions comprising Formula (I), or analog(s) thereof, are effective for treating hyperpigmentation disorders, such that the compounds, or analog(s) thereof, and/or compositions of the disclosure comprising such compounds, or analog(s) thereof, are used as skin tone-brightening or -evening treatments. Other embodiments provide for compounds and compositions comprising such compounds, or analog(s) thereof, (e.g., Formula (I)) of the disclosure, for use in treating a subject suffering from melanoma, either alone or in combination with commonly used therapies (e.g., chemotherapy; radiotherapy). Non-limiting examples of diseases or conditions associated with the production of melanin in the subject include melanogenesis dysfunction or dysregulation, vitiligo, melasma, hyperpigmentation, hypopigmentation, hypomelanic macules, Addison's disease, solar lentigo, senile lentigo, and skin cancers, such as melanoma.

### Pharmaceutical Compositions

The compounds described herein, or analog(s) thereof, (e.g., compounds, or analog(s) thereof, having the structure of Formula (I)), as well as, enantiomers, diastereomers, or a mixture of enantiomers and/or diastereomers (e.g., racemic mixture) thereof, or a pharmaceutically acceptable salt thereof, are useful alone or in physiologically- or pharmaceutically-acceptable compositions comprising such compounds, or analog(s) thereof. In some embodiments, compositions comprising any of the compounds described herein, or analog(s) thereof (e.g., Formula (I) as well as, enantiomers, diastereomers, or a mixture of enantiomers and/or diastereomers (e.g., racemic mixture) thereof, or a pharmaceutically acceptable salt thereof) are useful for the treatment of skin cancer, such as melanoma, or melanomas that arise from the skin, but develop in other locations, in a subject in need thereof. Additional embodiments provide for compositions comprising the compounds described herein, or analog(s) thereof, (e.g., Formula (I)) as a skin care formulation for brightening of a subject's skin tone, or the treatment of a pigmentation-related disorder, such as vitiligo or hyperpigmentation. In addition to the aforementioned diseases or conditions associated with the production of melanin in a subject, additional non-limiting examples include melanogenesis dysfunction or dysregulation, melasma, hypopigmentation, hypomelanic macules, Addison's disease, solar lentigo, and senile lentigo. Any of the compositions comprising compounds described here, or analog(s) thereof, can be used in a therapeutically effective amount to inhibit tyrosinase (i.e., function or regulation) or its activity, inhibit melanogenesis, inhibit melanin production, even out skin pigmentation or skin tone, brighten skin pigmentation or skin tone, inhibit cell proliferation of melanomas, ameliorate or treat skin cancer (e.g., melanoma), where the formulation is formulated for topical, oral, or parenteral administration.

Pharmaceutical dosage forms are provided as well, which can comprise a compound of the present disclosure, or analog(s) thereof, (e.g., compounds having the structure of Formula (I), or analog(s) thereof, as well as, enantiomers, diastereomers, or a mixture of enantiomers and/or diastereomers (e.g., racemic mixture) thereof, or a pharmaceutically acceptable salt thereof) and one or more pharmaceutically acceptable carriers, diluents, or excipients.

Unit dosage forms, also referred to as unitary dosage forms, often denote those forms of medication supplied in a manner that does not require further weighing or measuring to provide the dosage (e.g., tablet, capsule, caplet). The compositions of the present disclosure can be present as unit dosage forms. For example, a unit dosage form can refer to a physically discrete unit suitable as a unitary dosage for human subjects and other species, each unit containing a predetermined quantity of active material calculated to produce the desired therapeutic effect, in association with any suitable pharmaceutical excipient or excipients. Exemplary, non-limiting unit dosage forms include a tablet (e.g., a chewable tablet), caplet, capsule (e.g., a hard capsule or a soft capsule), lozenge, film, strip, and gel cap. In certain embodiments, the compounds described herein, or analog(s) thereof, including crystallized forms, polymorphs, and solvates thereof, can be present in a unit dosage form.

Useful pharmaceutical carriers, excipients, and diluents for the preparation of the compositions hereof, can be solids, liquids, or gases. These include any and all solvents, dispersion media, coatings, antibacterial and antifungal agents, isotonic and absorption delaying agents and the like. The pharmaceutically acceptable carrier or excipient does not destroy the pharmacological activity of the disclosed compound, or analog(s) thereof, and is nontoxic when administered in doses sufficient to deliver a therapeutic amount of the compound, or analog(s) thereof, or composition comprising the compound, or analog(s) thereof. Thus, the compositions can take the form of tablets, pills, capsules, suppositories, powders, enterically coated or other protected formulations (e.g., binding on ion-exchange resins or packaging in lipid-protein vesicles), sustained release formulations, topical formulations (e.g., lotion, gel, cream, ointment, paste), solutions, suspensions, elixirs, and aerosols. The carrier can be selected from the various oils including those of petroleum, animal, vegetable, or synthetic origin, e.g., peanut oil, soybean oil, mineral oil, and sesame oil. Water, saline, aqueous dextrose, and glycols are examples of liquid carriers, particularly (when isotonic with the blood) for injectable solutions. For example, formulations for intravenous administration comprise sterile aqueous solutions of the active ingredient(s), such as the compound described here, or analog(s) thereof, which are prepared by dissolving solid active ingredient(s) in water to produce an aqueous solution and rendering the solution sterile. Suitable pharmaceutical excipients include starch, cellulose, chitosan, tale, glucose, lactose, gelatin, malt, rice, flour, chalk, silica, magnesium stearate, sodium stearate, glycerol monostearate, sodium chloride, dried skim milk, glycerol, propylene glycol, water, and ethanol. The compositions can be subjected to conventional pharmaceutical additives such as preservatives, stabilizing agents, wetting or emulsifying agents, salts for adjusting osmotic pressure, and buffers. Suitable pharmaceutical carriers and their formulation are described in Remington's Pharmaceutical Sciences by E. W. Martin. Such compositions will, in any event, contain an effective amount of the active compound together with a suitable carrier so as to prepare the proper dosage form for administration to the recipient subject.

Any of the compositions described here comprising ML233, or analog(s) thereof, can further comprise physiologically acceptable, pharmaceutically acceptable, dermatologically acceptable, or cosmetically acceptable vehicles, carriers, diluents, active ingredients, or the like, which do not negatively affect the activity of the compound, or analog(s) thereof. Non-limiting examples of pharmaceutically acceptable carriers and excipients include sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; tale; cocoa butter and suppository waxes; oils such as peanut oil, cottonseed oil, safflower oil, sesame oil, olive oil, corn oil and soybean oil; glycols, such as polyethylene glycol and propylene glycol; esters such as ethyl oleate and ethyl laurate; agar; buffering agents such as magnesium hydroxide and aluminum hydroxide; alginic acid; isotonic saline; Ringer's solution; ethyl alcohol; phosphate buffer solutions; non-toxic compatible lubricants such as sodium lauryl sulfate and magnesium stearate; coloring agents; releasing agents; coating agents; sweetening, flavoring and perfuming agents; preservatives; antioxidants; ion exchangers; alumina; aluminum stearate; lecithin; self-emulsifying drug delivery systems (SEDDS) such as d-atocopherol polyethyleneglycol 1000 succinate; surfactants used in pharmaceutical dosage forms such as Tweens or other similar polymeric delivery matrices; serum proteins such as human serum albumin; glycine; sorbic acid; potassium sorbate; partial glyceride mixtures of saturated vegetable fatty acids; water, salts or electrolytes such as protamine sulfate, disodium hydrogen phosphate, potassium hydrogen phosphate, sodium chloride, and zinc salts; colloidal silica; magnesium trisilicate; polyvinyl pyrrolidone; cellulose-based substances; polyacrylates; waxes; and polyethylene-polyoxypropylene-block polymers. Cyclodextrins such as α-, β-, and γ-cyclodextrin, or chemically modified derivatives such as hydroxyalkylcyclodextrins, including 2- and 3-hydroxypropyl-cyclodextrins, or other solubilized derivatives can also be used to enhance delivery of the compounds described herein, or analog(s) thereof.

The pharmaceutical composition can also be formulated as a veterinary composition, intended for use with subjects other than humans. The veterinary compositions according to the present disclosure can be in any appropriate forms to suit the requested administration modes, for instance topical, nasal, oral, intradermic, cutaneous or parenteral. In a certain embodiment, the composition is in a form intended for oral administration and, for instance when the domestic animal eats, the composition described here is either mixed into the food ration, or directly into the mouth of the recipient before, during, or after a meal. The veterinary compositions of the disclosure are in the form of a nasal, oral or injectable liquid suspension or solution, or in solid or semi-solid form, powders, pellets, capsules, granules, sugar-coated pills, capsule, gelules, sprays, cachets, pills, tablets, lotions, creams, ointments, pastes, implants or gels. In one embodiment, the compositions are in the form of an oral solid form including tablets. In some embodiments, the veterinary compositions can have an effective amount of the compound for a specific species of animal (e.g., cow, lamb, goat, horse). In another embodiment, the compositions described here are in the form of a topical formulation, which includes gels, lotions, creams, ointments, pastes, sprays or aerosols, and the like.

In various embodiments, the compositions of the disclosure are formulated in pellets or tablets for an oral administration. According to this type of formulation, they comprise lactose monohydrate, cellulose microcrystalline, crospovidone/povidone, aroma, compressible sugar and magnesium stearate as excipients. When the compositions are in the form of pellets or tablets, they are for instance 1 mg, 2 mg, or 4 mg pellets or tablets. Such pellets or tablets are divisible so that they can be cut to suit the posology according to the disclosure in one or two daily takes. In a further embodiment, the compositions of the disclosure are formulated in injectable solutions or suspensions for a parenteral administration. The injectable compositions are produced by mixing therapeutically efficient quantity of torasemide with a pH regulator, a buffer agent, a suspension agent, a solubilization agent, a stabilizer, a tonicity agent and/or a preservative, and by transformation of the mixture into an intravenous, sub-cutaneous, intramuscular injection or perfusion according to a conventional method. Possibly, the injectable compositions can be lyophilized according to a conventional method. Examples of suspension agents include methylcellulose, polysorbate 80, hydroxyethylcellulose, xanthan gum, sodic carboxymethylcellulose and polyethoxylated sorbitan monolaurate. Examples of solubilization agent include polyoxy ethylene-solidified castor oil, polysorbate 80, nicotinamide, polyethoxylated sorbitan monolaurate, macrogol and ethyl ester of caste oil fatty acid. Moreover, the stabilizer includes sodium sulfite, sodium metalsulfite and ether, while the preservative includes methyl p-hydroxybenzoate, ethyl p-hydroxybenzoate, sorbic acid, phenol, cresol and chlorocresol. An example of tonicity agent is mannitol. When preparing injectable suspensions or solutions, it is desirable to make sure that they are blood isotonic.

In some embodiments, the pharmaceutical composition further comprises a viscosity enhancing agent. In some embodiments, the viscosity enhancing agent includes methylcellulose, hydroxyethylcellulose, hydroxypropylmethylcellulose and smart hydrogel. In some embodiments, the viscosity enhancing agent is hydroxyethylcellulose. In some embodiments, the pharmaceutical composition comprises 0.01-1.0% (w/v) viscosity enhancing agent. In other embodiments, the intranasal pharmaceutical composition comprises 0.05% (w/v) hydroxyethylcellulose.

In some embodiments, the pH of the pharmaceutical composition is from 4.0 to 7.5. In other embodiments, the pH of the pharmaceutical composition is from 4.0 to 6.5. In another embodiment the pharmaceutical composition has a pH of from 5.5 to 6.5. In further embodiments, the pharmaceutical composition has a pH of from 6.0 to 6.5. In various implementations, the pH of said aqueous solution or liquid formulation is from pH 3 to pH 7, from pH 3 to pH 6, from pH 4 to pH 6, or from pH 5 to pH 6. These pH ranges can be achieved through the incorporation of one or more pH modifying agents, buffers, and the like. In some embodiments, a pH modifier such as acetic acid, is present in a final concentration of at least about 0.001%, at least about 0.01%, or between about 0.01%-0.2% by weight of the composition.

In terms of their form, compositions of this disclosure can include solutions, emulsions (including microemulsions), suspensions, creams, lotions, pastes, ointments, gels, powders, aerosols, sprays, or other typical solid or liquid compositions used for application to skin and other tissues where the compositions can be used. Such compositions can contain: additional antimicrobials, moisturizers and hydration agents, penetration agents, preservatives, emulsifiers, natural or synthetic oils, solvents, surfactants, detergents, gelling agents, emollients, antioxidants, fragrances, fillers, thickeners, waxes, odor absorbers, dyestuffs, coloring agents, powders, viscosity-controlling agents and water, and optionally including anesthetics, anti-itch actives, botanical extracts, conditioning agents, darkening or brightening agents, glitter, humectants, mica, minerals, polyphenols, silicones or derivatives thereof, sunblocks, vitamins, and phytomedicinals. In certain embodiments, the composition of the disclosure is formulated with the above ingredients so as to be stable for a long period of time, and can be beneficial where continual or long-term treatment is intended.

### Methods of Using the Compound or Compositions

In some embodiments of the disclosure, the compound of Formula (I) (ML233), or analog(s) thereof, is used to inhibit tyrosinase and/or melanin production in a cell (e.g., melanocytes, tissue, subject). Described here are methods of inhibiting tyrosinase in a cell (in vitro, in vivo, ex vivo) comprises contacting the cell with the compound of Formula (I), i.e., ML233, or analog(s) thereof, which in turn inhibits tyrosinase in the cell. Additional embodiments of the disclosure provide for methods of inhibiting melanin production in a cell (in vitro, in vivo, ex vivo), where the methods comprise contacting the cell with ML233, or analog(s) thereof, thereby inhibiting melanin production in the cell. In some aspects, contacting can be by topical, oral, or parenteral administration, including but not limited to, intradermal, transdermal, intralesional, subcutaneous, intramuscular, and intravenous. Such methods of inhibiting tyrosinase and/or melanin production in a cell include cells that express tyrosinase and/or melanin. An exemplary cell type is a melanocyte. In addition to the methods being in vitro, in vivo, or ex vivo methods, similarly the cells can be in vitro, in vivo, or ex vivo. One of the cells for treatment includes melanocytes, tissue comprised of melanocytes, or melanocytes of a subject.

In further embodiments, any of the described skin care or pharmaceutical compositions comprising Formula (I), or analog(s) thereof, where Formula (I) or analog(s) thereof, is in a therapeutically effective amount having a concentration of 0.001 μM or greater (e.g., 0.005; 0.01; 0.05; 0.1; 0.15; 0.2; 0.25; 0.3; 0.35; 0.4; 0.45; 0.5; 0.55; 0.6; 0.65; 0.675; 0.725; 0.775; 0.825; 0.875; 0.925; 0.975; 1.025; 1.075; 1.125; 1.175; 1.225; 1.275; 1.325; 1.375; 1.425; 1.475; 1.525; 1.575; 1.625; 1.675; 1.725; 1.775; 1.825; 1.875; 1.925; 1.975; 2.025; 2.05; 2.25; 2.75; 3.25; 3.75; 4.25; 4.75; 5.25; 5.75; 6.25; 6.75; 7.25; 7.75; 8.25; 8.75; 9.25; 9.75; 10.25; 10.75; 11.25; 11.75; 12.25; 12.75; 13.25; 13.75; 14.25; 14.75; 15.25; 15.75; 16.25; 16.75; 17.25; 17.75; 18.25; 18.75; 19.25; 19.75; 20.25; 20.75; 21.25; 21.75; 22.25; 22.75; 23.25; 23.75; 24.25; 24.75; 25.25; 25.75; 26.25; 26.75; 27.25; 27.75; 28.25; 28.75; 29.25; 29.75; 30.25; 30.5; 30.75; 31; 31.25; 31.5; 31.75; 32); 30 μM or less (e.g., 29.5; 29; 28.5; 28; 27.5; 27; 26.5; 26; 25.5; 25; 24.5; 24; 23.5; 23; 22.5; 22; 21.5; 21; 20.5; 20.5; 20; 19.5; 19; 18.5; 18; 17.5; 17; 16.5; 16; 15.5; 15; 14.5; 14; 13.5; 13; 12.5; 12; 11.5; 11; 10.5; 10; 9.5; 9; 8.5; 8; 7.5; 7; 6.5; 6; 5.5; 5; 4.5; 4; 3.5; 3; 2.5; 2; 1.5; 1; 0.75; 0.5; 0.25; 0.175; 0.125; 0.058; 0.053; 0.045; 0.04; 0.35; 0.03; 0.025; 0.02; 0.015; 0.01; 0.005; 0.002); or a range of 0.001 μM-30 μM (e.g., 0.005 μM-29.8 μM; 0.01 μM-29.7; 0.05 μM-29.6 μM; 0.65 μM-29.5 μM; 0.675 μM-29 μM; 0.7 μM-28.75 μM; 0.725 μM-28.5 M; 0.75 μM-28.25 μM; 0.775 μM-28 μM; 0.8 μM-27.75 μM; 0.825 μM-27.5 M; 0.85 μM-27.25 μM; 0.875 μM-27 μM; 0.9 μM-26.725 μM; 0.925 μM-26.5 μM; 0.95 μM-26.25 μM; 0.975 μM-26 μM), thereby inhibiting tyrosinase (i.e., function or regulation) or its activity, inhibiting melanogenesis, inhibiting melanin production, evening out skin pigmentation or skin tone, brightening skin pigmentation or skin tone, inhibiting cell proliferation of melanomas, ameliorating or treating skin cancer (e.g., melanoma), where the formulation is formulated for topical, oral, or parenteral administration.

### Methods for Skin Tone Brightening

Some individuals are desirous of reducing pigmentation and evening skin tone or skin pigmentation. For example, individuals seek treatments for conditions such as hyperpigmentation and vitiligo. Additional embodiments of the disclosure provide a compound of Formula (I), or analog(s) thereof, or an enantiomer, a diastereomer, or a mixture of enantiomers and/or diastereomers (e.g., racemic mixture) thereof, or a pharmaceutically acceptable salt thereof, and compositions comprising such compounds or analog(s) thereof, which reduce melanogenesis, as well as inhibit tyrosinase function and/or activity, inhibit melanogenesis, inhibit melanin production, for reducing pigmentation or skin tone, brightening skin pigmentation or skin tone, and evening out skin pigmentation or skin tone.

In some embodiments, the compounds and compositions comprising such compounds described here, or analog(s) thereof, (e.g., Formula (I)) can be used to brighten hyperpigmentation of skin in a subject, reduce the appearance of uneven skin tone, reduce or improve uneven skin tone, and increase skin brightness. For administration of compounds, or analog(s) thereof, and compositions described herein, any mode of administration common or standard in the art may be used, e.g. topical, transdermal, oral, or intravenous delivery. In some aspects of the present disclosure, the compounds, or analog(s) thereof, and compositions can be prepared as a liquid, serum, salve, lotion, cream, gel, ointment, aerosol, spray, or emulsion.

Additional embodiments are directed to method for treating a disease or condition associated with the production of melanin in a subject, where the method comprises administering to the subject, a compound of formula (I) or ML233, or analog(s) thereof, or a composition comprising the compound of formula (I) or ML233, or analog(s) thereof, thereby treating the disease. In some aspects, the disease is associated with an undesirable increase in or high level of pigmentation in a cell, tissue, or a subject. Non-limiting examples of such diseases having an undesirable increase in or level of pigmentation in a cell, tissue, or a subject include: Addison's disease, hyperpigmentation, melasma, and solar lentigo. Other aspects provide for the disease associated with an undesirable decrease in or low level of pigmentation in a cell, tissue, or a subject. Without being bound by theory, the compound or composition of the disclosure decreases the melanin or pigmentation of the surrounding areas that are not affected by an undesirable decrease in or low level of pigmentation, thereby evening out the pigmentation. Non-limiting examples of such diseases having an undesirable decrease in or low level of pigmentation in a cell, tissue, or a subject include vitiligo and hypopigmentation. For example, a subject having vitiligo has some areas of hypopigmentation adjacent to pigmented skin. In order to even out the skin tone of a subject, the pigmented skin surrounding the hypopigmented areas are treated with a compound of formula (I) or ML233 to reduce the pigmentation or melanin. Similarly, methods for evening (e.g., lightening, brightening, or reducing) skin pigmentation in a subject, comprises administering to the subject a compound of formula (ML233) or a composition comprising ML233), which in turn lightens, brightens, or reduces skin pigmentation in the subject, thereby evening skin pigmentation or skin tone with respect to the surrounding areas of skin.

In some embodiments, a compound of Formula (I), or analog(s) thereof, can be administered as part of a skin care or cosmetic formulation. In some embodiments, a compound of Formula (I), or analog(s) thereof, can be included in a skin care or cosmetic vehicle. Examples of such skin care or cosmetic vehicles include creams, lotions, serums, pastes, lipsticks, gels, ointments, aerosols, sprays, and powders.

### Methods for Skin Cancer Treatment

In some embodiments, the compounds, or analog(s) thereof, and compositions comprising such compounds described here, or analog(s) thereof, (e.g., Formula (I), or analog(s) thereof, as well as, enantiomers, diastereomers, or a mixture of enantiomers and/or diastereomers (e.g., racemic mixture) thereof, or a pharmaceutically acceptable salt thereof) is used to treat skin cancers, such as melanoma. Melanomas also include mucosal melanoma (which develops in the mucous membrane of, for example, the nose, mouth, esophagus, anus, urinary tract, and vagina), ocular melanoma (which typically occurs in the uvea layer beneath the white of the eye), and acral-lentiginous melanoma (which typically occurs under a fingernail or a toenail, or on the palms of the hands or soles of the feet). It has been shown that some skin cancers, such as melanomas, are highly resistant to commonly used therapies such as radiation and chemotherapy. Additionally, the presence of melanin in metastatic melanoma cells has been linked to a decrease in successful outcomes in subjects receiving radiotherapy. Therefore, a compound of Formula (I), or analog(s) thereof, or an enantiomer, a diastereomer, or a mixture of enantiomers and/or diastereomers (e.g., racemic mixture) thereof, or a pharmaceutically acceptable salt thereof, and compositions comprising such compounds, or analog(s) thereof, which reduces melanogenesis, as well as inhibits tyrosinase activity and melanin production, are useful in treating such skin cancers. Provided here are methods of treating a skin cancer (e.g., melanoma) in a subject in need thereof, comprising: administering a compound of Formula (I), or analog(s) thereof, or an enantiomer, a diastereomer, or a mixture of enantiomers and/or diastereomers (e.g., racemic mixture) thereof, or a pharmaceutically acceptable salt thereof to the subject in need thereof.

In some embodiments, the methods of treating a skin cancer in a subject in need thereof includes administering a composition comprising a compound of Formula (I), or analog(s) thereof, or an enantiomer, a diastereomer, or a mixture of enantiomers and/or diastereomers (e.g., racemic mixture) thereof, or a pharmaceutically acceptable salt thereof, and a vehicle, such as a physiologically- or pharmaceutically-acceptable vehicle (e.g., carrier, diluent, excipient) to the subject suffering from skin cancer; these compositions can be formulated for topical, oral, parenteral, or intravenous delivery.

Typically, the treatment of a disease, disorder, or condition (e.g., the conditions described herein such as those associated with cells expressing tyrosinase, cells expressing melanin, melanocytes, neoplastic cells, and the like, or combinations thereof, is an approach for obtaining beneficial or desired results, such as clinical and or therapeutic results. Beneficial or desired results can include, but are not limited to, alleviation or amelioration of one or more symptoms or conditions; diminishment of extent of disease, disorder, or condition; stabilized (i.e., not worsening) state of disease, disorder, or condition; preventing spread of disease, disorder, or condition; delay or slowing the progress of the disease, disorder, or condition; amelioration or palliation of the disease, disorder, or condition; and remission (whether partial or total), whether detectable or undetectable. A disease, disorder, or condition can be palliated which includes that the extent and/or undesirable clinical manifestations of the disease, disorder, or condition are lessened and/or time course of the progression is slowed or lengthened, as compared to the extent or time course in the absence of treatment.

In order to treat, prevent, or prevent recurrence of diseases, disorders, or conditions (e.g., melanoma, vitiligo, hyperpigmentation) as discussed herein, the compounds, or analog(s) thereof, or compositions comprising such compounds or analog(s) thereof of the present disclosure can be administered at least once a day for at least one week. In various embodiments, the composition is administered at least twice a day for at least two days. In certain embodiments, the composition is administered approximately daily, at least daily, twice a week, weekly, or for once a month. In certain embodiments, the composition of the disclosure is administered for several months, such as at least two months, six months, or one year or longer, or for a sufficient amount of time to see a beneficial and/or therapeutic effect. The disclosure is further suited for long-term use, which can be particularly beneficial for preventing recurring hyperpigmentation or to maintain even skin tone. Such long-term use can involve treatment for at least two years, three years, four years, or even five or more years, as can be determined by one of skill in the art, or the subject's clinician.

### Combination Therapies

Accordingly, in some embodiments, the compounds, or analog(s) thereof, and pharmaceutical compositions comprising such compounds, or analog(s) thereof, (e.g., Formula (I)) described here can be formulated and employed in combination therapies. Some aspects of the embodiment provide compounds, or analog(s) thereof, and pharmaceutical compositions described here, that can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed can achieve a desired effect for the same disorder, or they can achieve different effects (e.g., control of any adverse effects).

Examples of other drugs to combine with the compounds, or analog(s) thereof, described herein include pharmaceuticals for the treatment of melanoma, such as, but not limited to, cisplatin, carboplatin, dacarbazine, paclitaxel, temozolomide, and vinblastine. Combination methods can involve the use of the two (or more) agents formulated together or separately, as determined to be appropriate. In one example, two or more drugs are formulated together for the simultaneous or near simultaneous administration of the agents. In other embodiments, the compounds of the disclosure, or analog(s) thereof, can be used in combination with other treatments, including but not limited to, chemotherapy, radiotherapy, immunotherapy, surgery, or the like, to treat a disease associated with the production of melanin, e.g., melanoma.

### Kits

In another aspect, a kit is provided here, which contains a compound of Formula (I) or ML233, or analog(s) thereof, as well as, enantiomers, diastereomers, or a mixture of enantiomers and/or diastereomers (e.g., racemic mixture) thereof, or a pharmaceutically acceptable salt thereof, packaged to facilitate dispensing and/or administration of the compositions disclosed herein. Some aspects provide instructions for use of the ML233 compounds, or analog(s) thereof, or compositions comprising such ML233 compounds, or analog(s) thereof, to inhibit melanogenesis, inhibit melanin production, inhibit tyrosinase, treat melanoma, to lighten or brighten skin pigmentation, and the like. The packaging or dispenser can include a bottle, tube, spray bottle, or other dispenser. In certain embodiments of the disclosure, the composition comprising any of the compounds of the disclosure, or analog(s) thereof, is packaged in a concentrated form, and diluted to a desired concentration upon use by the end user. Typically, in these aspects, such compositions can be formulated and packaged in a manner suitable for long-term storage to maintain efficacy of the composition.

The practice of the present disclosure employs, unless otherwise indicated, conventional techniques of chemistry, molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of or testing of compounds of the disclosure, or analog(s) thereof, or their effectiveness, and, as such, can be considered in making and practicing the disclosure. Useful techniques for particular embodiments are discussed in the sections that follow.

The following examples are put forth so as to provide those of ordinary skill in the art with a complete disclosure and description of how to make and use the assay, screening, and therapeutic methods of the disclosure, and are not intended to limit the scope of what the inventors regard as their disclosure.

### EXAMPLES

### Example 1: Inhibition of Skin Pigmentation Via ML233

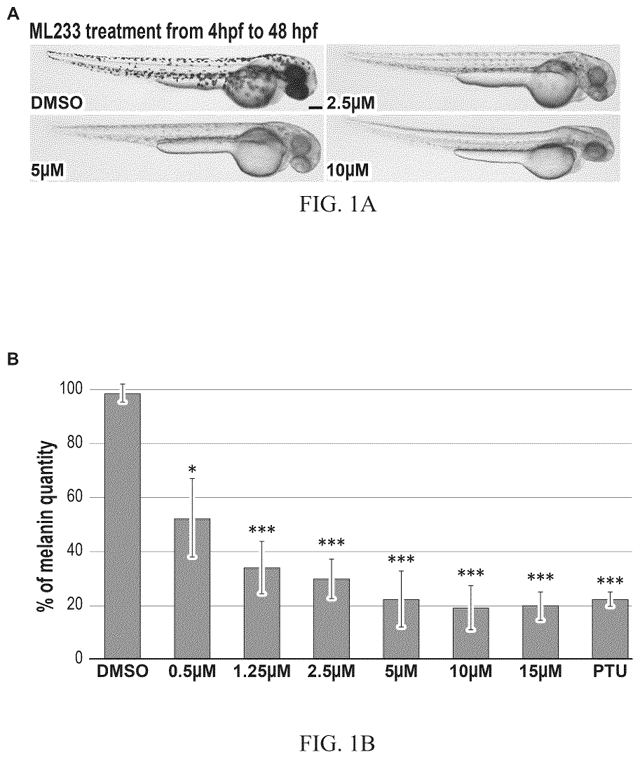

Zebrafish embryos were allowed to develop in the presence of ML233 and the effects of ML233 on pigmentation was analyzed. ML233 was dissolved in carrier Dimethylsulfoxide (DMSO). Embryos were exposed to ML233 beginning at four hours post-fertilization and ending at 48 hours post-fertilization. Dimethylsulfoxide (DMSO) was used as a negative control. ML233 dosages tested were 2.5 micromolar, five micromolar, and 10 micromolar (FIG. 1A). A visible decrease in pigmentation was seen in embryos treated with ML233 compared to the negative control. These results show that ML233 treatment, even at lower concentrations, reduced pigmentation.

The effect of ML233 on percent melanin quantity was assayed in zebrafish embryos beginning at 4 hours post-fertilization and ending at 48 hours. Developing embryos were treated with ML233 in DMSO carrier at varying dosages, or DMSO as a negative control or with phenylthiourea (PTU) as a positive control. Concentrations of ML233 administered were: 0.5 micromolar, 1.25 micromolar, 2.5 micromolar, five micromolar, 10 micromolar, and 15 micromolar (FIG. 1B). ML233 significantly inhibited melanin production at all levels tested. Effects of ML233 administration at 5 micromolar and above were comparable to treatment with PTU, a potent tyrosinase inhibitor. This indicates that ML233 is a potent inhibitor of melanogenesis.

### Example 2: ML233 Reduces Melanin Production

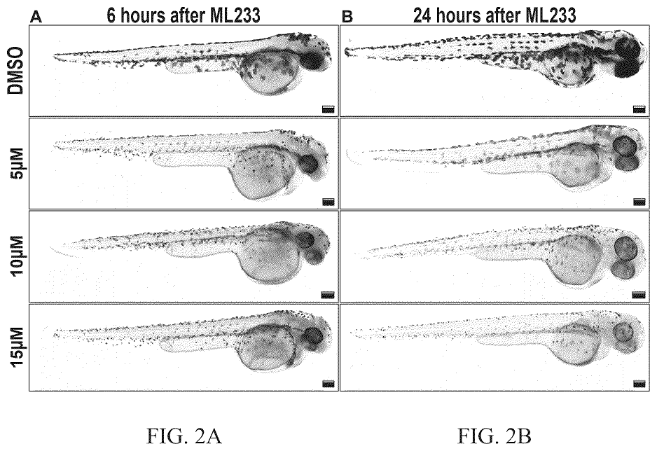

The effect of ML233 on pigmentation was analyzed in zebrafish embryos at 38 hours post-fertilization when larvae were highly pigmented. Embryos were treated with ML233 at the following concentrations: 5 micromolar, 10 micromolar, and 15 micromolar. The carrier, DMSO, was used as a negative control. Pigmentation was analyzed six hours (FIG. 2A) and 24 hours after ML233 addition (FIG. 2B). Treatment with ML233 reduced pigmentation at all dosages.

### Example 3: ML233 is a Chemical Inhibitor of Tyrosinase Activity

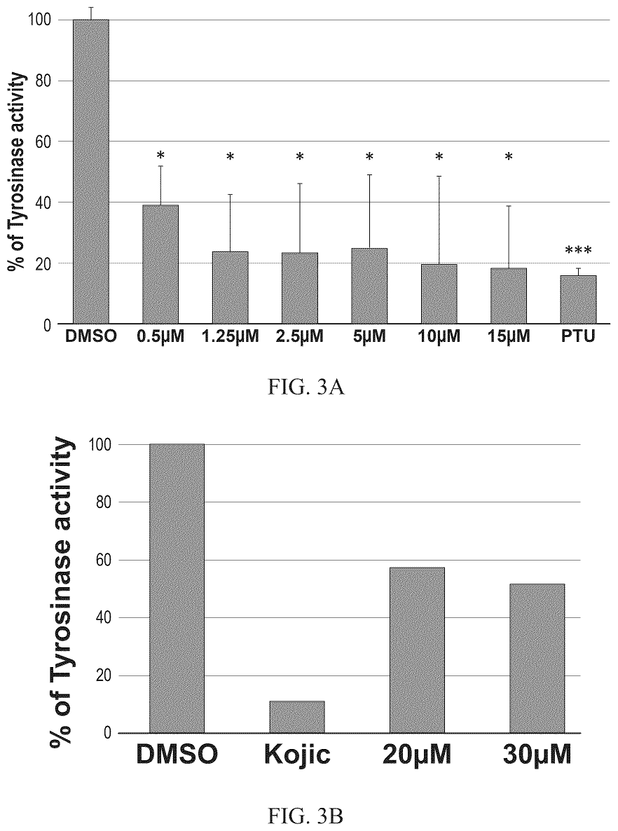

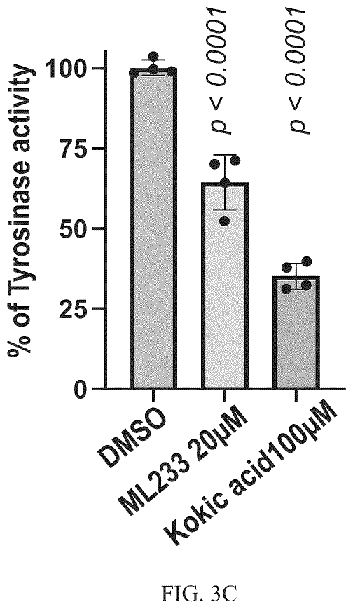

To determine the effect of ML233 on tyrosinase activity, zebrafish embryos were treated with ML233 at the following concentrations: 0.5 micromolar, 1.25 micromolar, 2.5 micromolar, five micromolar, 10 micromolar, and 15 micromolar ML233 (FIG. 3A). DMSO was used as a carrier and the tyrosinase inhibitor PTU was used as a positive control. The percent of tyrosinase activity was determined with spectrophotometric absorbance of L-DOPA and found to be comparable to PTU in the 10 micromolar and 15 micromolar ML233 groups, indicating that like PTU ML233 strongly inhibits tyrosinase activity.

The effect of ML233 on tyrosinase activity was also explored in in vitro experiments. The percent of tyrosinase activity was drastically reduced in response ML233 treatment resulted in strongly reduced tyrosinase activity compared to DMSO, with greater than 40% reduction of tyrosinase activity in the 20 micromolar ML233 addition group, and 50% reduction of tyrosinase activity in the 30 micromolar ML233 addition group (FIG. 3B). Tyrosinase activity was significantly reduced with a concentration of ML233 as low as 0.5 micromolar.

To determine whether ML233, a small inhibitor of melanogenesis, acts directly on tyrosinase (TYR) activity in vitro, the activity of mushroom TYR was assessed via measurement of its capacity to convert L-DOPA to melanin in vitro using Kojic Acid at 100 μM as a positive control. Quantification of ML233-mediated melanogenesis inhibition revealed a significant reduction of melanogenesis by ˜40% in samples treated with 20 μM of ML233 compared to DMSO (negative control)-only-treated samples (FIG. 3C). This in vitro experiment confirmed in vivo observations regarding ML233-mediated inhibition of TYR activity and suggested that this small chemical inhibitor of melanogenesis acts directly on the TYR protein to regulate its function.

### Example 4: Comparative Toxicity of ML233 at Effective Concentrations

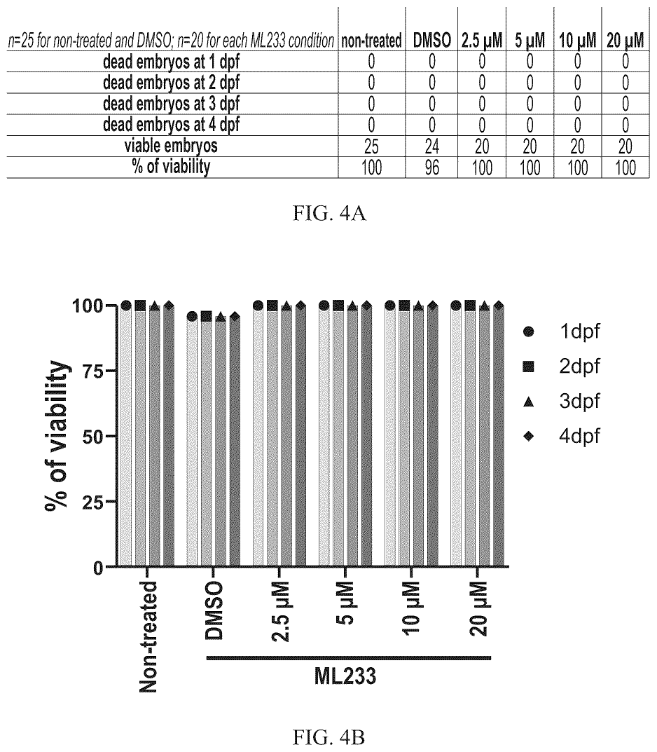

Studies were undertaken to evaluate the effect of ML233 on embryo viability. Embryos at 4 hours post-fertilization were treated with varying doses of ML233: 2.5 micromolar, five micromolar, 10 micromolar, and 20 micromolar of ML233, and the effects were evaluated. The number of viable and dead embryos at 1 day post fertilization (dpf), 2 dpf, 3 dpf and 4 dpf and at various doses of ML233 was compared to embryo viability in negative controls, which included embryos treated with the carrier DMSO or untreated embryos. (FIG. 4A). This indicated that ML233 has low toxicity at effective concentrations.

Bar chart representation of viability, zebrafish embryos were treated with 2.5 micromolar, five micromolar, 10 micromolar and 20 micromolar ML233 and percent viability was determined at day four post fertilization for ML233 treated fish and negative controls that were untreated or treated with DMSO carrier alone (FIG. 4B). No deleterious effects on zebrafish viability were observed.

### Example 5: Effects of ML233 on Mammalian Cells

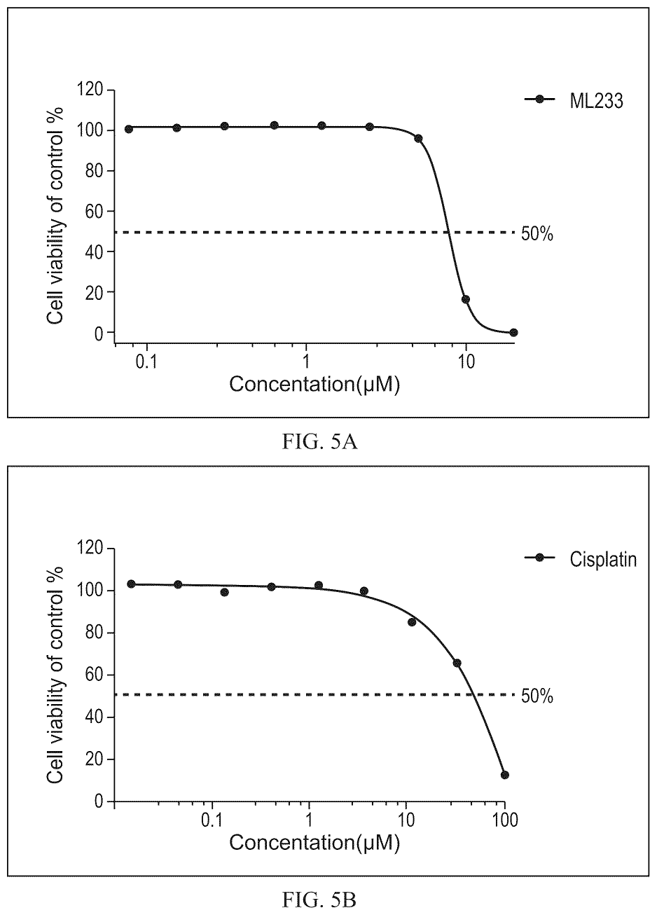

ML233 reduced pigmentation in a mammalian melanoma cell line. In vivo, in vitro, and computational studies described here supported the hypothesis that ML233 was a direct inhibitor of TYR protein expression and function. The effects of ML233 and cisplatin on the proliferation of B16F-10 murine melanoma cells in vitro was analyzed. To determine the effects of conservation in higher vertebrate species, ML233 activity in the B16F10 murine melanoma cell line was tested. B16F10-cell proliferation was analyzed after ML233 treatment to determine IC50. A 50% reduction in cell viability at concentrations of ML233 between 5 and 10 μM was observed (FIG. 5A). Cells were treated with various concentration of ML233 or cisplatin and the cell viability of control percent versus the concentration of ML233 or cisplatin (in μM) was analyzed (FIGS. 5A and 5B). The in vitro IC50 of ML233 was determined to be 10 micromolar, which was at least three times lower than the observed IC50 of cisplatin. Comparison of the effect of ML233 with that of cisplatin treatment (FIGS. 5A-5B) suggested a potent inhibition of melanoma-cell proliferation by ML233.

### Example 6: ML233 Reduced Melanogenesis in Melanoma Cells

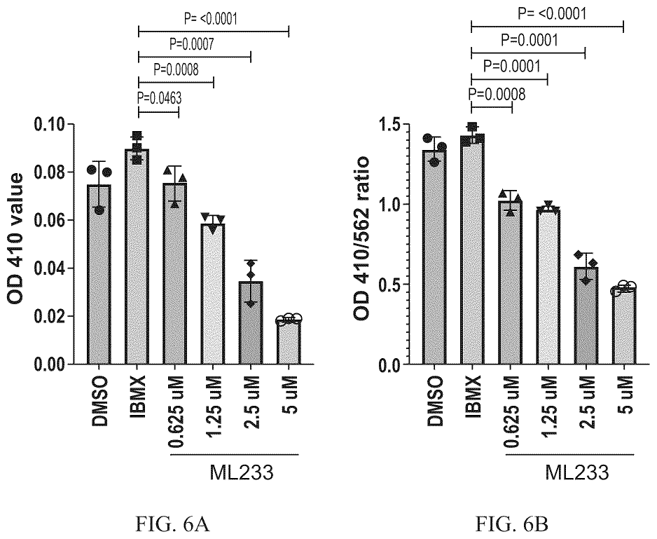

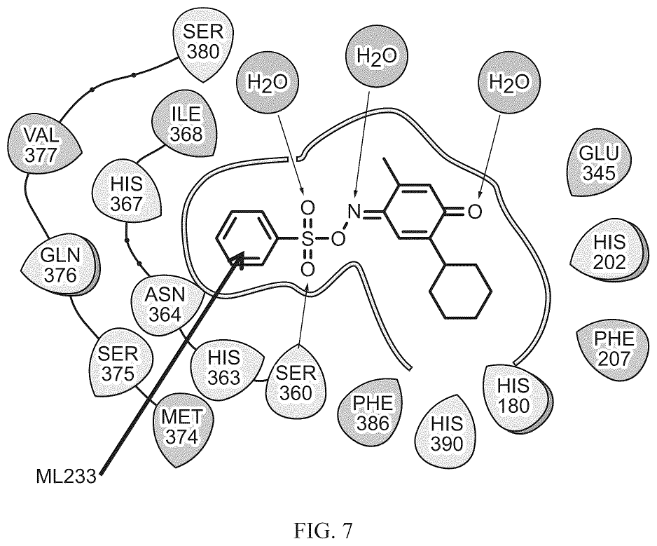

To determine if ML233 could inhibit pigmentation of B16F20 cells at low ML233 concentrations (without effects on cell proliferation), melanin production was analyzed in B16F10 cells after 24 hours of treatment with 0.625 μM, 1.25 μM, 2.5 μM, or 5 μM of ML233. Observation of cell pellets after centrifugation confirmed that ML233 function to inhibit melanin production was conserved in mammals (data not shown). The effect of ML233 and 3-isobutyl-1-methylxanthine (IBMX) on melanin production was analyzed in B16F10 murine melanoma cells. IBMX is a non-selective phosphodiesterase inhibitor, which induced melanin production in B16F10 murine melanoma cells. Melanin production was measured in cells treated with IBMX alone (100 micromolar), and IBMX in conjunction with: 0.625 micromolar ML233, 1.25 micromolar ML233, 2.5 micromolar ML233, and five micromolar ML233, as well as in negative control cells treated with DMSO vehicle alone. Melanin production in treated cells was measured using optical density (FIG. 6A) at a wavelength of 410 nm (OD value 410). Ratios with the total quantity of protein (OD value 562) were calculated (FIG. 6B). ML233 reduced melanogenesis in mammalian melanoma cells at all dosages. P-values are indicated in the bar charts.

Absorbance quantification of melanin confirmed this observation and revealed that concentrations of ML233 as low as 0.625 μM significantly reduced melanoma-cell pigmentation (FIGS. 6A-6B). Concentrations between 0.625 and 5 μM of ML233 had the capacity to reduce melanin production without affecting B16F10 cells survival. Together, these results confirmed data and validated ML233 as a potent inhibitor of melanogenesis in mammalian cells.

Inhibition of melanin production was also observed in ML233 analogs: (i) [(Z)-(5-methyl-4-oxo-2-propan-2-ylcyclohexa-2,5-dien-1-ylidene)amino]benzenesulfonate (Compound CID: 5731374; PubChem SID: 437251522; (ii) CSSS00159739712); [(E)-(5-methyl-4-oxo-2-propan-2-ylcyclohexa-2,5-dien-1-ylidene)amino]4-methylbenzenesulfonate (Compound CID: 5731427; PubChem SID: 437714551; CSSS00160289749); and (iii) [(E)-(2-methyl-4-oxo-5-propan-2-ylcyclohexa-2,5-dien-1-ylidene)amino]benzenesulfonate (Compound CID: 5786741; PubChem SID: 437912457; CSSS00160623089). The ML233 analogs were tested in parallel with serial dilution in DMSO: at 25 μM, 12.5 μM, 6.25 M, 3.12 5 μM, 1.5625 μM and 0.78125 RM. All of the compounds showed an efficient and potent activity to inhibit melanin production, starting at a concentration of 0.78125 μM. ML233 analogs (i), (ii), and (iii) were found to have higher toxicity when tested at 25 μM as compared to ML233. ML233 did not have detectable toxicity at 25 μM. ML233 was found to be very efficient at inhibiting melanin production at 3.125 M and lower concentrations.

### Example 7: Molecular Docking of TYR-ML233 Interaction

Molecular docking analysis of TYR-ML233 was performed in order to predict the potential binding site of ML233 with human TYR tyrosinase (FIG. 7). ML233 was found to have a potential binding site with a small pocket in the tyrosinase protein, suggesting that binding at this site may allow for a stable interaction between the tyrosinase protein and ML233 at that location.

### Example 8: Prediction of ML233 Binding in the Active Site of the Human Tyrosinase Protein by Molecular Docking

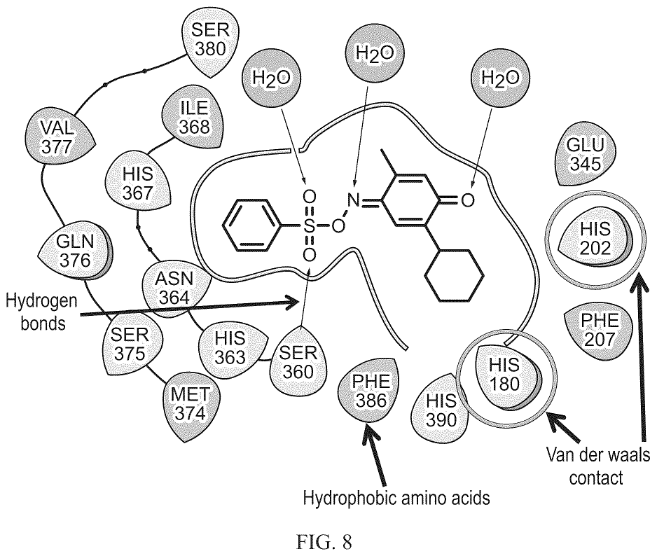

Molecular docking of TYR-ML233 was performed in order to determine potential binding sites of ML233 to TYR (FIG. 8). This 2D representation of TYR and ML233 interacting showed hydrogen bonds between ML233 and Ser360 and each of the three water molecules (H₂O). Amino acids had different properties: hydrophobic amino acids (Phe207, Ile368, Met374, Val377, Phe386); polar amino acids (His180, His202, Ser360, His363, Asn364, His367, Ser375, Gln376, Ser380, His390); and acidic negatively charged amino acid (Glu345). By identifying potential interactions between ML233 and His202 and His180, which regulated tyrosinase activity, the pocket identified in the above example was correctly determined to be the active site of the TYR protein. Therefore, ML233 inhibited TYR protein activity by occupying copper ion binding sites.

### Example 9: Tyrosinase Expression after ML233 Treatment

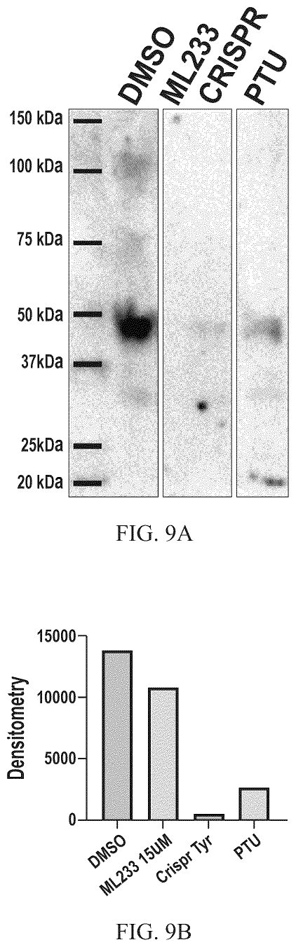

An analysis of tyrosinase expression after ML233 treatment was performed. A Western blot demonstrated that after 15 μM of ML233, tyrosinase expression was inhibited (FIG. 9A). Quantitative analysis was also performed using densitometry of tyrosinase expression after ML233 treatment at 15 μM of the Western blot data (FIG. 9B). DMSO was a negative control, while phenylthiourea (PTU), which is a potent inhibitor of human tyrosinase), was used as a positive control. CRISPR was used as a specificity control of the antibody used in the analysis, where CRISPR was directed against the tyrosinase gene.

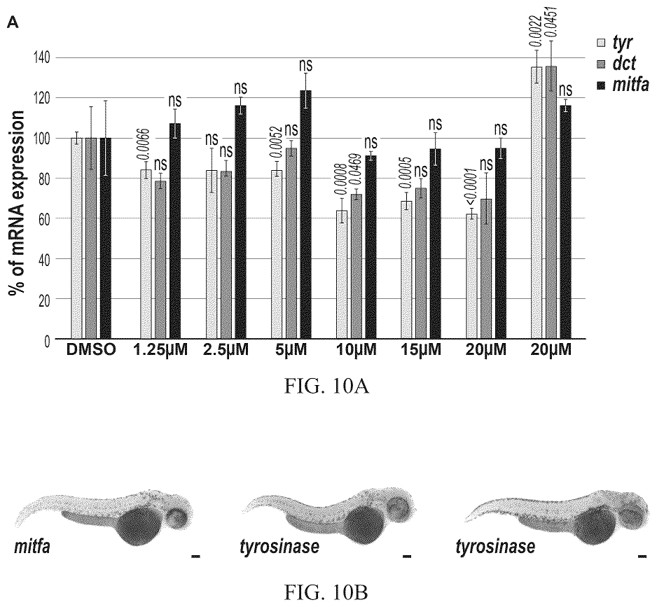

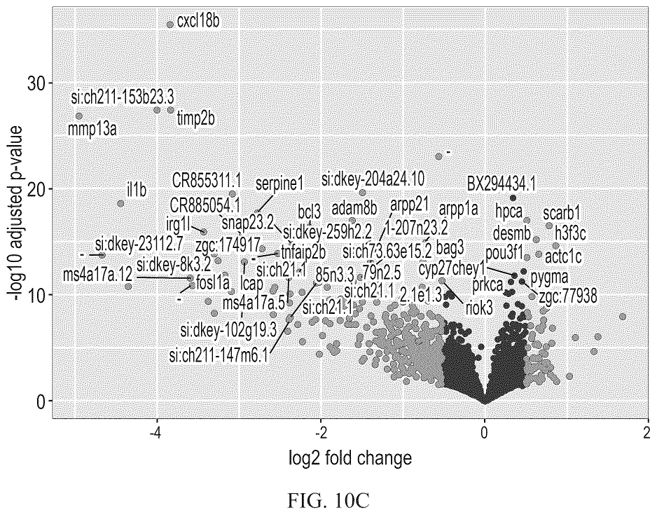

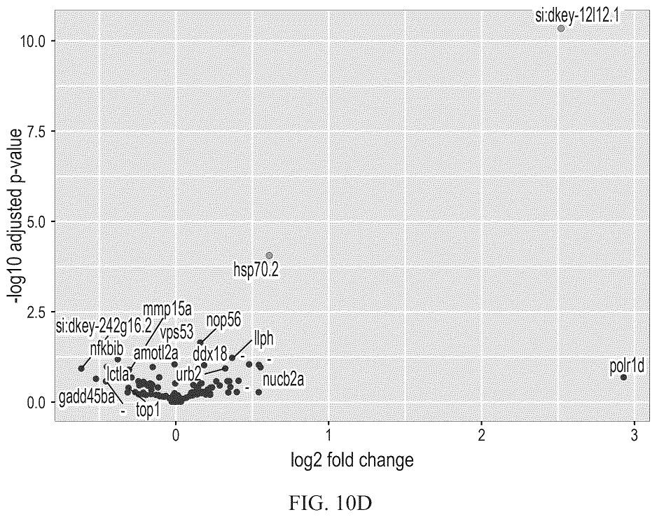

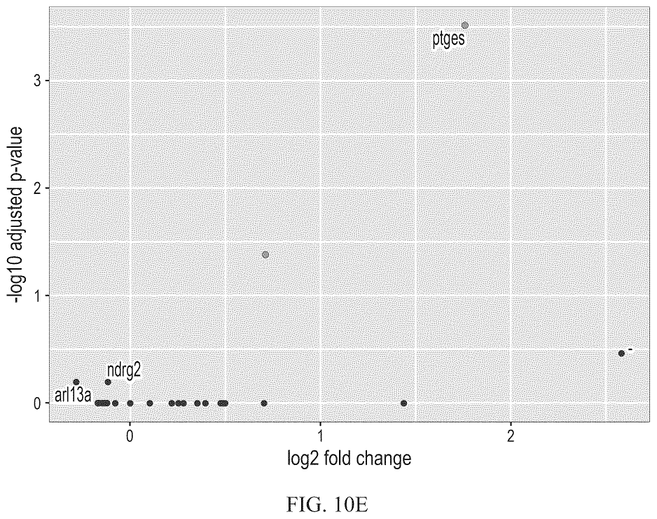

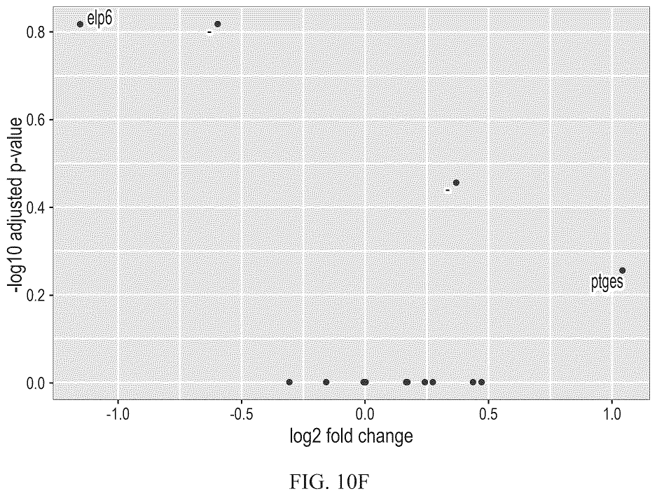

Example 10: Tyrosinase gene expression was not abolished by ML233 treatmentTo better understand the molecular action of ML233 on skin pigmentation and melanogenesis, the expression of genes involved in melanogenesis, including tyrosinase (tyr), dopachrome tautomerase (dct), and microphthalmia-associated transcription factor (mitfa), the latter which is known to control melanocyte formation and TYR expression, was analyzed. Global quantification of tyr, dct, and mitfa mRNA expression by RT-qPCR in zebrafish embryos at 2 dpf indicated a mild reduction of gene expression after 1 day of ML233 treatment (FIG. 10A). Significantly decreased tyr expression was observed at ML233 concentrations of 5 μM and greater, with a maximum reduction of ˜40% at 20 μM of ML2333 compared to control DMSO-treated embryos. Expression of the transcription factor mitfa, master regulator of melanocyte formation and melanogenesis, was not significantly dysregulated by ML233 treatment at any concentration. In addition, in situ hybridization demonstrated that the mitfa and tyr genes were still expressed in melanocytes at 2 dpf after 1 day of 15 μM ML233 treatment (FIG. 10B; left, center). Similarly to PTU-treated embryos (FIG. 10B; right), tyr mRNA is highly detectable in ML233-treated embryos in a pattern resembling that of melanocyte skin cell organization. These results supported observations indicating that melanocytes were still present in the skin after ML233 treatment despite the lack of melanin production.

Because ML233 is an agonist of the apelin receptors, ML233 driven differential expression in an apelin dependent and independent manner was investigated. Zebrafish embryos were treated with 0.5 μM of ML233 (a concentration showed to significantly reduce melanin content and TYR protein activity, FIG. 1B and FIG. 3A) for 3 hours or 24 hours in wild-type (WT) or apelin receptors Knock-out (aplnr KO) genetic background. A clear separation in gene expression between the WT and KO backgrounds was observed (FIG. 10C). However, minimal expression changes were identified for either WT or KO within the genotype for both the 3 h and 24 h exposure conditions (FIGS. 10D-10F), indicating that genotype variation (WT vs KO) was the strongest determinant underlying change in gene expression profiles.

Gene Set Enrichment Analysis (GSEA) indicated that ML233 treatment at this concentration did not significantly impact biological processes such as, but not limited to, inflammation, stress response or apoptosis (data not shown), further indicating ML233 to be a potentially tolerable pharmaceutical chemical.

### Example 11: Tyrosinase Protein Expression was not Affected by ML233 Treatment

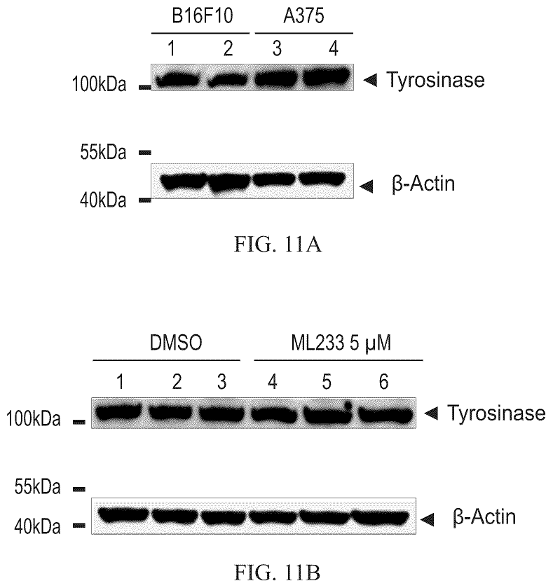

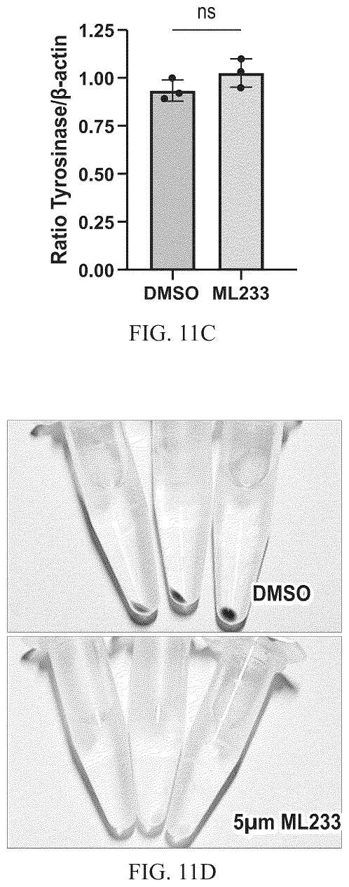

Because tyrosinase mRNA expression was not dramatically reduced after ML233 treatment in vivo and because in vitro data suggested that ML233 could directly inhibit the Tyrosinase protein function, a reduction in skin pigmentation that reflected either a reduction of the TYR protein expression and/or an inhibition of the protein enzymatic activity was hypothesized. To study these two possibilities, an antibody raised against the human tyrosinase protein was used. The capacity of the antibody to recognize TYR in two different mammalian cell lines (FIG. 11A): B16F10 (murine melanoma) and A375 (human melanoma) was tested. Using the validated antibody, the level of tyrosinase protein expression by Western blot in control DMSO and 5 μM ML233 treated cells (FIG. 11B) was analyzed. There was no significant reduction of TYR protein expression normalized with Beta-actin protein expression (FIG. 11C). In the same cells and at the same concentration of ML233 treatment, a strong reduction in the pigmentation was also observed (FIG. 11D).

### Example 12: Computational Prediction of ML233-Dependent Direct Inhibition of Tyrosinase Protein Function

The structural relationship between ML233 and the TYR protein was studied to better understand the potential mechanisms underlying ML233-dependent inhibition of TYR activity. The protein structure of human TYR was predicted by AlphaFold2 (data not shown). The derivative 3D structure was used to establish the predictive conformation. Five potential asparagine (N)N-glycosylation sites were identified in TYR: N86, N111, N230, N290, and N371. Molecular docking experiments were conducted to predict the optimal binding site of ML233 within the TYR protein (see, e.g., FIGS. 7-8). This analysis indicated that ML233 could bind the ligand-binding pocket of the human TYR protein.

The molecular dynamics and binding energy between the protein and the small chemical inhibitor were also analyzed. The dynamic simulation (10 ns-100 ns) showed that the overall protein structure was relatively stable and that the binding site of the small molecule did not change during the simulation (data not shown). The hydrophobic and hydrophilic areas changed following ML233 binding during the binding simulation. The simulation (10 ns-100 ns) showed only a subtle change in the distribution (data not shown), indicating that the small molecule binding had a small effect on the hydrophilic and hydrophobic domains. ML233-TYR binding stability was quantified using Root Mean Square Deviation (RMSD) analysis (data not shown). The RMSD value remained lower than 2 Å throughout the simulation, maintaining an average level of 1.54 Å. An RMSD value below 2 Å suggested no drastic structural change, predicting that the TYR-ML233 interaction and the protein structure were relatively stable. The amino acids showing the greatest amount of time of contact with ML233 during the dynamic simulation were His180, Glu345, Ser360, His367, Ile368, and Val377 (see, FIGS. 7-8), suggesting that these were the most important amino acids for binding of ML233 on the TYR protein.

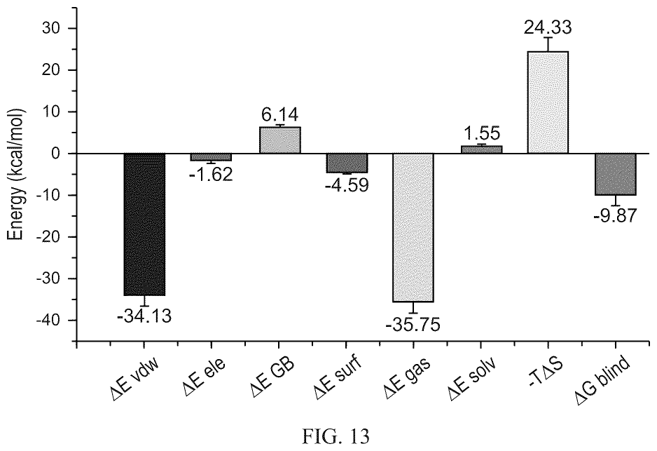

The binding free energy between ML233 and TYR was also quantified and analyzed (FIG. 13), which revealed a binding free energy of −9.87 kcal/mol and a dissociation constant of 58.18 nmol was calculated (Kd=ΔG/RT). This strong binding affinity was primarily the result of the van der Waals action (ΔEvdw), which was −34.13 kcal/mol, together with the almost negligible electrostatic effect (ΔEele), which was only −1.62 kcal/mol. The free energy of polar solvents (ΔEGB) was 6.14 kcal/mol, and the free energy of non-polar solvents (ΔEsurf) was −4.59 kcal/mol, indicating that the total free energy (ΔGsolv) is 1.55 kcal/mol and that less energy would be lost in desolvation. Because no drastic change in protein structure was observed during the dynamic simulation, the potential entropy loss (−TΔS=24.33 kcal/mol) of the system was hypothesized to be due to subtle structural adjustments and oscillations of the small molecule itself at the binding site.

The mode of interaction between ML233 and the TYR protein is represented in FIGS. 7-8. The sulfoxide group of the ML233 molecule has oxygen atoms that formed a 2.9 Å hydrogen bond with Ser360. Another oxygen atom in the sulfide group and the unsaturated N atom formed 2.4 Å and 2.1 Å hydrogen bonds, respectively, with water molecules. The carbonyl oxygen atom on the molecular benzene ring also formed a 1.9 Å hydrogen bond with a water molecule. In addition, the benzene ring of the molecule formed hydrophobic interactions with Ile368 and Val377, and polar van der Waals interactions with His367, Ser380, Gln376, and other polar amino acids. The molecular cyclohexane had hydrophobic interactions with the hydrophobic amino acids Phe207 and Phe386 and van der Waals contacts with the polar amino acids His180, His202 and His390.

Histidines (His180, His202, His367 and His390) on the human TYR protein were proposed to regulate TYR activity by binding copper ions (I. Kampatsikas & A. Rompel. Chembiochem 22, 1161-1175, 2021; Spritz et al. J Invest Dermatol 109, 207-212, 1997; Noh, et al. JEnzyme Inhib Med Chem 35, 726-732, 2020). The above computational analysis suggested that a stable interaction between ML233 and TYR in the functional site of the protein could occupy copper-ion binding sites and inhibit TYR enzymatic activity. This mechanism of inhibition supported in vivo and in vitro data regarding the inhibition of TYR activity by ML233.

### Example 13: ML233 Inhibited Proliferation of PDX-Derived Melanoma Organoids

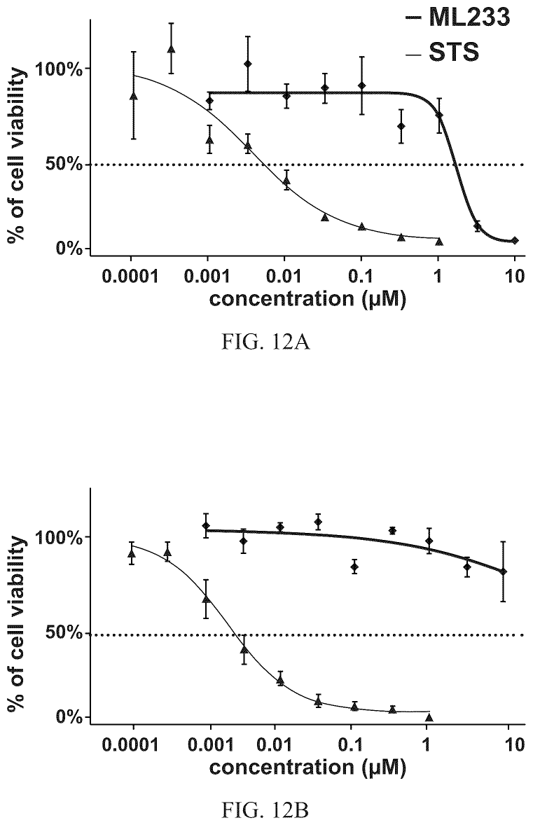

Because ML233 affected B16F10 murine melanoma-cell proliferation, the proliferative potential of human metastatic melanoma was determined. Patient-derived xenograft organoids (PDXOs), which were 3D in vitro models generated from patient tumors, were used. The effect of ML233 treatment (between 0.001 μM and 10 μM) in two different human melanoma cell lines collected from metastasis, ME1154B and ME2319B (Crown Bioscience). These two melanoma lines differed in their sensitivity to both ML233 and the control molecule, staurosporine (STS), in 3D organoids (FIGS. 12A-12B). ML233 inhibited ME1154B viability/proliferation (IC50=1.65 μM) (FIG. 12A, right curve), but did not affect ME2319B viability at any concentration tested (FIG. 12B, right curve). However, ME2319B was more sensitive (IC50=0.0025 μM) (FIG. 12B, left/lower curve) than ME1154B (IC50=0.0054 μM) (FIG. 12A, left/lower curve) to STS treatment. Together, these data indicated that ML233 could inhibit proliferation of human metastatic melanoma in PDXO in vitro models. This observation suggested that ML233 could be tested in PDX organoids or mice as an alternative to other therapeutics showing limitations in the treatment of melanoma.

### OTHER EMBODIMENTS

From the foregoing description, it will be apparent that variations and modifications can be made to the disclosure described herein to adopt it to various usages and conditions. Such embodiments are also within the scope of the following claims.

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A method of inhibiting tyrosinase in a cell, the method comprising: contacting the cell with ML233, or analog(s) thereof, thereby inhibiting tyrosinase in the cell.

2. A method of inhibiting melanin production in a cell, the method comprising: contacting the cell with ML233, or analog(s) thereof, thereby inhibiting melanin production in the cell.

3. The method of claim 1, wherein the cell is a cell expressing tyrosinase and/or melanin.

4. The method of claim 1, wherein the cell is a melanocyte.

5. The method of claim 1, wherein the cell is a cell in vivo or in vitro.

6. The method of claim 1, wherein the cell is a neoplastic cell.

7. The method of claim 1, wherein the contacting is by topical or parenteral administration.

8. A method for treating a disease associated with the production of melanin in a subject, comprising administering to the subject a composition comprising ML233, or analog(s) thereof, thereby treating the disease.

9. The method of claim 8, wherein the disease is associated with an undesirable increase in pigmentation.

10. The method of claim 9, wherein the disease is selected from the group consisting of Addison's disease, hyperpigmentation, melasma, and solar lentigo.

11. The method of claim 8, wherein the disease is associated with an undesirable decrease in pigmentation.

12. The method of claim 11, wherein the disease is vitiligo or hypopigmentation.

13. The method of claim 8, wherein the composition is formulated for topical, oral, or intravenous delivery.

14. A method for treating melanoma, the method comprising contacting the cells with ML233, or analog(s) thereof, thereby treating the melanoma.

15. The method of claim 14, wherein the method further comprises administrating a chemotherapy drug or treating the subject in need thereof with radiation treatment.

16. A method for evening skin pigmentation in a subject, the method comprising administering to the subject a composition comprising ML233, or analog(s) thereof, thereby evening skin pigmentation in the subject.

17. The method of claim 16, wherein the composition further comprises one or more physiologically acceptable carriers.

18. The method of claim 1, wherein the analog(s) thereof is selected from the group consisting of:
[(Z)-(5-methyl-4-oxo-2-propan-2-ylcyclohexa-2,5-dien-1-ylidene)amino]benzenesulfonate (Compound CID: 5731374; PubChem SID: 43 7251522; CSSS00159739712);
[(E)-(5-methyl-4-oxo-2-propan-2-ylcyclohexa-2,5-dien-1-ylidene)amino]4-methylbenzenesulfonate (Compound CID: 5731427; PubChem SID: 437714551;CSSS00160289749);
[(E)-(2-methyl-4-oxo-5-propan-2-ylcyclohexa-2,5-dien-1-ylidene)amino]benzenesulfonate (Compound CID: 5 786741; PubChem SID: 43791245 7; CSSS00160623089);
[(Z)-(5-cyclohexyl-2-methyl-4-oxocyclohexa-2,5-dien- 1-ylidene)amino]benzenesulfonate (Compound CID: 46905036);
[(Z)-(2-methyl-4-oxo-5-propan-2-ylcyclohexa-2,5-dien-1-ylidene)amino]benzenesulfonate (Compound CID: 5601088);
[(E)-(5-methyl-4-oxo-2-propan-2-ylcyclohexa-2,5-dien-1-ylidene)amino]benzenesulfonate (Compound CID: 5650290);
[[4-oxo-3,5-di(propan-2-yl)cyclohexa-2,5-dien-1-ylidene]amino]benzenesulfonate (Compound CID: 5258778);
[(Z)-(5-methyl-4-oxo-2-propan-2-ylcyclohexa-2,5-dien-1-ylidene)amino]4-methylbenzenesulfonate (Compound CID: 5941439);
[(2-methyl-4-oxo-5-propan-2-ylcyclohexa-2,5-dien-1-ylidene)amino]benzenesulfonate (Compound CID: 1909670);
[(5-methyl-4-oxo-2-propan-2-ylcyclohexa-2,5-dien-1-ylidene)amino]benzenesulfonate (Compound CID: 222113 8);
[(Z)-(2-methyl-4-oxo-5-propan-2-ylcyclohexa-2,5-dien-1-ylidene)amino]4-propan-2-ylbenzenesulfonate (Compound CID: 49787116);
[(E)-(5-cyclohexyl-2-methyl-4-oxocyclohexa-2;5-dien-1-ylidene)amino]3-chlorobenzenesulfonate (Compound CID: 49852493);
[(Z)-(5-cyclohexyl-2-methyl-4-oxocyclohexa-2;5-dien-1-ylidene)amino]3-methylbenzenesulfonate (Compound CID: 50898262);
[(Z)-(2-methyl-4-oxo-5-propan-2-ylcyclohexa-2,5-dien-1-ylidene)amino]3-methylbenzenesulfonate (Compound CID: 50898263);
[(Z)-(5-cyclohexyl-2-methyl-4-oxocyclohexa-2;5-dien-1-ylidene)amino]3-bromobenzenesulfonate (Compound CID: 50898264);
[(E)-(5-cyclohexyl-2-methyl-4-oxocyclohexa-2;5-dien-1-ylidene)amino]2-chlorobenzenesulfonate (Compound CID: 50898268);
[(Z)-(5-cyclohexyl-2-methyl-4-oxocyclohexa-2;5-dien-1-ylidene)amino]3-cyanobenzenesulfonate (Compound CID: 50898270);
(NZ)-N-[(4aR,8aS)-6,7-dimethyl-4-oxo-4a,5,8,8a-tetrahydronaphthalen-1-ylidene]benzenesulfonamide (Compound CID: 134879088);
[(4-oxo-3-phenylcyclohexa-2,5-dien- 1-ylidene) amino]benzenesulfonate (Compound CID: 50898267);
[(E)-(4-oxo-4-phenylbutan-2-ylidene)amino]benzenesulfonate (Compound CID: 88531954);
[(E)-(4-oxo-5-phenylpentan-2-ylidene)amino]benzenesulfonate (Compound CID: 88532017);
[(E)-(5-oxo-5-phenylpentan-2-ylidene)amino]benzenesulfonate (Compound CID: 88532051);
[(E)-(1-oxo-1-phenylpropan-2-ylidene)amino]benzenesulfonate (Compound CID: 88532204);
[(E)-(1-oxo-1-phenylpentan-3-ylidene)amino]benzenesulfonate (Compound CID: 88532276) ;
[(4-oxo-1-phenylpentan-2-ylidene)amino]benzenesulfonate (Compound CID: 88532489);
[(2,6-ditert-butyl-4,5-dioxocycloheX-2-en-1-ylidene)amino]benzenesulfonate (Compound CID: 123626753);
[(5-methyl-4-oxo-2-propan-2-ylcyclohexa-2,5-dien-1-ylidene)amino]4-methylbenzenesulfonate (Compound CID: 29065 67);
[(2-methyl-4-oxo-5-propan-2-ylcyclohexa-2,5-dien-1-ylidene)amino]4-methylbenzenesulfonate (Compound CID: 5245 197);
[(E)-(2-methyl-4-oxo-5-propan-2-ylcyclohexa-2,5-dien-1-ylidene)amino]4-methylbenzenesulfonate (Compound CID: 6311908);
[(Z)-(2-methyl-4-oxo-5-propan-2-ylcyclohexa-2,5-dien-1-ylidene)amino]4-methylbenzenesulfonate (Compound CID: 6536676); and
[(5-cyclohexyl-2-methyl-4-oxocyclohexa-2,5-dien-1-ylidene)amino]benzenesulfonate (Compound CID: 75249343).

19. A kit for use in the method of claim 1, wherein the kit comprises ML233, or analog(s) thereof.

20. The kit of claim 19, wherein the kit further comprises instructions for using ML233, or analog(s) thereof, to inhibit melanogenesis and/or treat melanoma.
